SEQUENCE LISTING (1 sequence)

SEQ ID NO: 1 - protein, 785 aa, Homo sapiens
  source 1..785: note = human USP1 protein
MPGVIPSESNGLSRGSPSKKNRLSLKFFQKKETKRALDFTDSQENEEKASEYRASEIDQVVPAAQSSPINCEKRENLLPFVGLNNLGNTCYLNSILQVLYFCPGFKSGVKHLFNIISRKKEALKDEANQKDKGNCKEDSLASYELICSLQSLIISVEQLQASFLLNPEKYTDELATQPRRLLNTLRELNPMYEGYLQHDAQEVLQCILGNIQETCQLLKKEEVKNVAELPTKVEEIPHPKEEMNGINSIEMDSMRHSEDFKEKLPKGNGKRKSDTEFGNMKKKVKLSKEHQSLEENQRQTRSKRKATSDTLESPPKIIPKYISENESPRPSQKKSRVKINWLKSATKQPSILSKFCSLGKITTNQGVKGQSKENECDPEEDLGKCESDNTTNGCGLESPGNTVTPVNVNEVKPINKGEEQIGFELVEKLFQGQLVLRTRCLECESLTERREDFQDISVPVQEDELSKVEESSEISPEPKTEMKTLRWAISQFASVERIVGEDKYFCENCHHYTEAERSLLFDKMPEVITIHLKCFAASGLEFDCYGGGLSKINTPLLTPLKLSLEEWSTKPTNDSYGLFAVVMHSGITISSGHYTASVKVTDLNSLELDKGNFVVDQMCEIGKPEPLNEEEARGVVENYNDEEVSIRVGGNTQPSKVLNKKNVEAIGLLGGQKSKADYELYNKASNPDKVASTAFAENRNSETSDTTGTHESDRNKESSDQTGINISGFENKISYVVQSLKEYEGKWLLFDDSEVKVTEEKDFLNSLSPSTSPTSTPYLLFYKKL

(19) United States
(12) Patent Application Publication
(10) Pub. No.: US 2025/0161306 A1
(43) Pub. Date: May 22, 2025

(54) UBIQUITIN-SPECIFIC-PROCESSING PROTEASE 1 (USP1) INHIBITORS FOR THE TREATMENT OF SOLID TUMORS

(71) Applicant: KSQ Therapeutics, Inc., Lexington, MA (US)

(72) Inventors: Andrew Alistair WYLIE, Newton, MA (US); Anne Louise CADZOW, Boston, MA (US); Hanlan LIU, Lexington, MA (US); Andrew James OLAHARSKI, Winchester, MA (US); Adrian SENDEROWICZ, Surfside, FL (US)

(21) Appl. No.: 18/729,657

(22) PCT Filed: Jan. 24, 2023
(86) PCT No.: PCT/US2023/061184

§ 371 Date: Jul. 17, 2024

Related U.S. Application Data
(60) Provisional application No. 63/302,891, filed on Jan. 25, 2022.
(60) Provisional application No. 63/375,601, filed on Sep. 14, 2022.
(60) Provisional application No. 63/384,554, filed on Nov. 21, 2022.

(51) Int. Cl. A61K 31/519

(52) U.S. Cl. CPC A61K 31/519

(57) ABSTRACT

The present disclosure provides a substituted pyrazolopyrimidine, 6-(4-cyclopropyl-6-methoxypyrimidin-5-yl)-1-(4-(1-isopropyl-4-(trifluoromethyl)-1 H-imidazol-2-yl)benzyl)-1H-pyrazolo[3,4-d]pyrimidine, or a salt, solvate, hydrate, or co-crystal thereof, as a USP1 inhibitor utilized in a method for use in treating a cancer, especially a method of treating a solid tumor in a human patient and therapeutically effective dosing regimen therefore that can elicit effective responses in humans while also avoiding dose-limiting adverse effects.

### CROSS-REFERENCE TO RELATED PATENT APPLICATIONS

This application claims priority from U.S. Provisional Application Nos. 63/302,891, filed Jan. 25, 2022, 63/375,601, filed Sep. 14, 2022, and 63/384,554, filed Nov. 21, 2022, each of which is incorporated herein by reference in its entirety.

### REFERENCE TO A SEQUENCE LISTING SUBMITTED ELECTRONICALLY

The content of the electronically submitted sequence listing (Name 4195_029PC03_Seqlisting_ST26; Size: 3,002 bytes; and Date of Creation: Jan. 20, 2023) is herein incorporated by reference in its entirety.

### FIELD OF THE INVENTION

The present disclosure relates to methods of using ubiquitin-specific-processing protease one (USP1) inhibitors to treat solid tumors and uses of USP1 inhibitors to treat solid tumors, wherein the USP1 inhibitors are provided in therapeutically effective amounts. Provided herein are therapeutically effective dosing regimens.

### BACKGROUND

Substituted pyrazolopyrimidines, such as 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine are inhibitors of USP1, and USP1 has been identified as a target in cancer therapy. 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine inhibitors can inhibit USP1 with low nM potency and is selective for USP1 over other de-ubiquitinase family members. Moreover, this USP1 inhibitor is selectively active in a specific subset of cell lines and not broadly cytotoxic. However, there is a critical need to identify particular dosage regimens of USP1 inhibitors that can elicit effective responses in humans while also avoiding dose-limiting adverse effects.

### BRIEF SUMMARY

Provided herein are dosing regimens for the administration of USP1 inhibitors for the treatment of solid tumors. For example, provided herein is a method of treating a solid tumor in a human patient, comprising administering to the patient about 75 mg to about 2500 mg of the ubiquitin-specific-processing protease 1 (USP1) inhibitor 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound I), or a salt, solvate, hydrate, or co-crystal thereof in an amount equivalent to about 75 mg to about 2500 mg of Compound I. Also provided herein is a method of treating a solid tumor in a human patient, comprising administering to the patient about 75 mg to about 1000 mg of the ubiquitin-specific-processing protease 1 (USP1) inhibitor 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound I), or a salt, solvate, hydrate, or co-crystal thereof in an amount equivalent to about 75 mg to about 1000 mg of Compound I. Also provided herein is a method of treating a solid tumor in a human patient, comprising administering to the patient about 1000 mg to about 2500 mg of the ubiquitin-specific-processing protease 1 (USP1) inhibitor Compound I, or a salt, solvate, hydrate, or co-crystal thereof in an amount equivalent to about 1000 mg to about 2500 mg of Compound I.

In some aspects, about 75 mg of the USP1 inhibitor or an amount of the salt, solvate, hydrate, or co-crystal thereof equivalent to about 75 mg of the USP1 inhibitor is administered. In some aspects, about 100 mg of the USP1 inhibitor or an amount of the salt, solvate, hydrate, or co-crystal thereof equivalent to about 100 mg of the USP1 inhibitor is administered. In some aspects, about 150 mg of the USP1 inhibitor or an amount of the salt, solvate, hydrate, or co-crystal thereof equivalent to about 150 mg of the USP1 inhibitor is administered. In some aspects, about 200 mg of the USP1 inhibitor or an amount of the salt, solvate, hydrate, or co-crystal thereof equivalent to about 200 mg of the USP1 inhibitor is administered. In some aspects, about 250 mg of the USP1 inhibitor or an amount of the salt, solvate, hydrate, or co-crystal thereof equivalent to about 250 mg of the USP1 inhibitor is administered. In some aspects, about 300 mg of the USP1 inhibitor or an amount of the salt, solvate, hydrate, or co-crystal thereof equivalent to about 300 mg of the USP1 inhibitor is administered. In some aspects, about 350 mg of the USP1 inhibitor or an amount of the salt, solvate, hydrate, or co-crystal thereof equivalent to about 350 mg of the USP1 inhibitor is administered. In some aspects, about 400 mg of the USP1 inhibitor or an amount of the salt, solvate, hydrate, or co-crystal thereof equivalent to about 400 mg of the USP1 inhibitor is administered. In some aspects, about 450 mg of the USP1 inhibitor or an amount of the salt, solvate, hydrate, or co-crystal thereof equivalent to about 450 mg of the USP1 inhibitor is administered. In some aspects, about 500 mg of the USP1 inhibitor or an amount of the salt, solvate, hydrate, or co-crystal thereof equivalent to about 500 mg of the USP1 inhibitor is administered. In some aspects, about 550 mg of the USP1 inhibitor or an amount of the salt, solvate, hydrate, or co-crystal thereof equivalent to about 550 mg of the USP1 inhibitor is administered. In some aspects, about 600 mg of the USP1 inhibitor or an amount of the salt, solvate, hydrate, or co-crystal thereof equivalent to about 600 mg of the USP1 inhibitor is administered. In some aspects, about 650 mg of the USP1 inhibitor or an amount of the salt, solvate, hydrate, or co-crystal thereof equivalent to about 650 mg of the USP1 inhibitor is administered. In some aspects, about 700 mg of the USP1 inhibitor or an amount of the salt, solvate, hydrate, or co-crystal thereof equivalent to about 700 mg of the USP1 inhibitor is administered. In some aspects, about 750 mg of the USP1 inhibitor or an amount of the salt, solvate, hydrate, or co-crystal thereof equivalent to about 750 mg of the USP1 inhibitor is administered. In some aspects, about 800 mg of the USP1 inhibitor or an amount of the salt, solvate, hydrate, or co-crystal thereof equivalent to about 800 mg of the USP1 inhibitor is administered. In some aspects, about 850 mg of the USP1 inhibitor or an amount of the salt, solvate, hydrate, or co-crystal thereof equivalent to about 850 mg of the USP1 inhibitor is administered. In some aspects, about 900 mg of the USP1 inhibitor or an amount of the salt, solvate, hydrate, or co-crystal thereof equivalent to about 900 mg of the USP1 inhibitor is administered. In some aspects, about 950 mg of the USP1 inhibitor or an amount of the salt, solvate, hydrate, or co-crystal thereof equivalent to about 950 mg of the USP1 inhibitor is administered. In some aspects, about 1000 mg of the USP1 inhibitor or an amount of the salt, solvate, hydrate, or co-crystal thereof equivalent to about 1000 mg of the USP1 inhibitor is administered. In some aspects, about 1050 mg of the USP1 inhibitor or an amount of the salt, solvate, hydrate, or co-crystal thereof equivalent to about 1050 mg of the USP1 inhibitor is administered. In some aspects, about 1100 mg of the USP1 inhibitor or an amount of the salt, solvate, hydrate, or co-crystal thereof equivalent to about 1100 mg of the USP1 inhibitor is administered. In some aspects, about 1150 mg of the USP1 inhibitor or an amount of the salt, solvate, hydrate, or co-crystal thereof equivalent to about 1150 mg of the USP1 inhibitor is administered. In some aspects, about 1120 mg of the USP1 inhibitor or an amount of the salt, solvate, hydrate, or co-crystal thereof equivalent to about 1200 mg of the USP1 inhibitor is administered. In some aspects, about 1250 mg of the USP1 inhibitor or an amount of the salt, solvate, hydrate, or co-crystal thereof equivalent to about 1250 mg of the USP1 inhibitor is administered. In some aspects, about 1300 mg of the USP1 inhibitor or an amount of the salt, solvate, hydrate, or co-crystal thereof equivalent to about 1300 mg of the USP1 inhibitor is administered. In some aspects, about 1350 mg of the USP1 inhibitor or an amount of the salt, solvate, hydrate, or co-crystal thereof equivalent to about 1350 mg of the USP1 inhibitor is administered. In some aspects, about 1400 mg of the USP1 inhibitor or an amount of the salt, solvate, hydrate, or co-crystal thereof equivalent to about 1400 mg of the USP1 inhibitor is administered. In some aspects, about 1450 mg of the USP1 inhibitor or an amount of the salt, solvate, hydrate, or co-crystal thereof equivalent to about 1450 mg of the USP1 inhibitor is administered. In some aspects, about 1500 mg of the USP1 inhibitor or an amount of the salt, solvate, hydrate, or co-crystal thereof equivalent to about 1500 mg of the USP1 inhibitor is administered. In some aspects, about 1550 mg of the USP1 inhibitor or an amount of the salt, solvate, hydrate, or co-crystal thereof equivalent to about 1550 mg of the USP1 inhibitor is administered. In some aspects, about 1600 mg of the USP1 inhibitor or an amount of the salt, solvate, hydrate, or co-crystal thereof equivalent to about 1600 mg of the USP1 inhibitor is administered. In some aspects, about 1650 mg of the USP1 inhibitor or an amount of the salt, solvate, hydrate, or co-crystal thereof equivalent to about 1650 mg of the USP1 inhibitor is administered. In some aspects, about 1700 mg of the USP1 inhibitor or an amount of the salt, solvate, hydrate, or co-crystal thereof equivalent to about 1700 mg of the USP1 inhibitor is administered. In some aspects, about 1750 mg of the USP1 inhibitor or an amount of the salt, solvate, hydrate, or co-crystal thereof equivalent to about 1750 mg of the USP1 inhibitor is administered. In some aspects, about 1800 mg of the USP1 inhibitor or an amount of the salt, solvate, hydrate, or co-crystal thereof equivalent to about 1800 mg of the USP1 inhibitor is administered. In some aspects, about 1850 mg of the USP1 inhibitor or an amount of the salt, solvate, hydrate, or co-crystal thereof equivalent to about 1850 mg of the USP1 inhibitor is administered. In some aspects, about 1900 mg of the USP1 inhibitor or an amount of the salt, solvate, hydrate, or co-crystal thereof equivalent to about 1900 mg of the USP1 inhibitor is administered. In some aspects, about 1950 mg of the USP1 inhibitor or an amount of the salt, solvate, hydrate, or co-crystal thereof equivalent to about 1950 mg of the USP1 inhibitor is administered. In some aspects, about 2000 mg of the USP1 inhibitor or an amount of the salt, solvate, hydrate, or co-crystal thereof equivalent to about 2000 mg of the USP1 inhibitor is administered. In some aspects, about 2100 mg of the USP1 inhibitor or an amount of the salt, solvate, hydrate, or co-crystal thereof equivalent to about 2100 mg of the USP1 inhibitor is administered. In some aspects, about 2200 mg of the USP1 inhibitor or an amount of the salt, solvate, hydrate, or co-crystal thereof equivalent to about 2200 mg of the USP1 inhibitor is administered. In some aspects, about 2300 mg of the USP1 inhibitor or an amount of the salt, solvate, hydrate, or co-crystal thereof equivalent to about 2300 mg of the USP1 inhibitor is administered. In some aspects, about 2400 mg of the USP1 inhibitor or an amount of the salt, solvate, hydrate, or co-crystal thereof equivalent to about 2400 mg of the USP1 inhibitor is administered. In some aspects, about 2500 mg of the USP1 inhibitor or an amount of the salt, solvate, hydrate, or co-crystal thereof equivalent to about 2500 mg of the USP1 inhibitor is administered.

In some aspects, about 75 mg, about 100 mg, about 150 mg, about 200 mg, about 250 mg, about 300 mg, about 350 mg, about 400 mg, about 450 mg, about 500 mg, about 550 mg, about 600 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2100 mg, about 2200 mg, about 2300 mg, about 2400 mg, about 2500 mg, or a range between any of the numbers recited in this paragraph of the USP1 inhibitor or an amount of the salt, solvate, hydrate, or co-crystal thereof equivalent to about 75 mg, about 100 mg, about 150 mg, about 200 mg, about 250 mg, about 300 mg, about 350 mg, about 400 mg, about 450 mg, about 500 mg, about 550 mg, about 600 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2100 mg, about 2200 mg, about 2300 mg, about 2400 mg, about 2500 mg, or a range between any of the numbers recited in this paragraph of the USP1 inhibitor is administered.

In some aspects, the USP1 inhibitor or the salt, solvate, hydrate, or co-crystal thereof is administered about once daily. In some aspects described in the paragraph above, the USP1 inhibitor is administered as a divided dose over the course of a day. For example, in some aspects, a the USP1 inhibitor is administered over a course of two administrations in a day (e.g., if the dose is 1000 mg or equivalents thereof, the USP1 inhibitor is administered in a first dose of 500 mg and a second dose of 500 mg on the same day, wherein the first dose and the second dose are not simultaneous). The amount of the first dose and the second dose may be the same or different.

In some aspects, the USP1 inhibitor or the salt, solvate, hydrate, or co-crystal thereof is administered orally.

In some aspects, the cancer is ovarian cancer. In some aspects, the ovarian cancer is serous ovarian cancer.

In some aspects, the cancer is breast cancer. In some aspects, the patient has previously received platinum-based chemotherapy. In some aspects, the cancer is platinum-sensitive. In some aspects, the cancer is platinum-resistant. In some aspects, the breast cancer is human epidermal growth factor receptor 2 (HER2) negative. In some aspects, the breast cancer is triple negative breast cancer.

In some aspects, the cancer has a mutation in BRCA1. In some aspects, the cancer has a mutation in BRCA2.

In some aspects, the breast cancer is germline breast cancer susceptibility gene mutation (gBRCAm) breast cancer. In some aspects, the breast cancer is metastatic.

In some aspects, the patient has previously received treatment with at least one systemic chemotherapy for metastatic disease. In some aspects, the cancer is selected from the group consisting of a DNA damage repair pathway deficient cancer, a homologous-recombination deficient cancer, a cancer comprising cancer cells with a mutation in a gene encoding p53, and a cancer comprising cancer cells with a loss of function mutation in a gene encoding p53. In some aspects, the cancer is selected from the group consisting of lung cancer, non-small cell lung cancer (NSCLC), colon cancer, bladder cancer, osteosarcoma, ovarian cancer, skin cancer, and breast cancer.

In some aspects, the cancer comprises cancer cells with elevated levels of RAD18 protein and/or RAD18 mRNA. In some aspects, the cancer comprises cancer cells with elevated levels of RAD51 protein and/or RAD51 mRNA. In some aspects, the elevated levels of RAD51 are elevated RAD51 protein foci levels. In some aspects, at least 10% of cells that are in the S/G2 phase of the cell cycle in a sample obtained from the cancer are RAD51-positive.

In some aspects, the cancer is recurrent. In some aspects, the patient has previously received treatment with a PARP inhibitor (PARPi). In some aspects, the cancer is a PARP inhibitor resistant or refractory cancer.

In some aspects, the Compound I is administered as a co-crystal with a pharmaceutically acceptable acid. In some aspects, the pharmaceutically acceptable acid is gentisic acid.

In some aspects, the USP1 inhibitor or the salt, solvate, hydrate, or co-crystal thereof is administered at least three times, at least 4 times, or at least 5 times, or at least times, or at least 15 times, or at least 20 days, or at least 25 days, or at least 30 days, or at least 60 days, or at least 90 days, or at least 120 days, or least 150 days, or at least 180 days, or at least 210 days, or at least 240 days, or at least 270 days, or at least 300 days, or at least 330 days, or at least 360 days, or at least 365 days, or more. In some aspects, the USP1 inhibitor or the salt, solvate, hydrate, or co-crystal thereof is administered for a period of at least 3 days, or at least 4 days, or at least 5 days, or at least 10 days, or at least 15 days, or at least 20 days, or at least 25 days, or at least 1 month, or at least 2 months, or at least 3 months, or at least 4 months, or at least 5 months, or at least 6 months, or at least 7 months, or at least 8 months, or at least 9 months, or at least 10 months, or at least 11 months, or at least 1 year, or at least 18 months, or at least 2 years, or more.

Also provided herein is a USP1 inhibitor Compound I or salt, solvate, hydrate, or co-crystal thereof for use in any method provided herein.

### BRIEF DESCRIPTION OF THE DRAWINGS/FIGURES

FIG. 1 is a powder X-ray diffraction pattern (“XRPD”) corresponding to crystalline Form A. (See Example 1.)

FIG. 2 is a differential scanning calorimetry thermogram (“DSC”) and a thermogravimetric analysis thermogram (“TGA”) corresponding to crystalline Form A. (See Example 1.)

FIG. 3 is an asymmetric unit of crystalline Form A from a single crystal structure. (See Example 1.)

FIG. 4 is an XRPD pattern corresponding to crystalline Form 2. (See Example 2.)

FIG. 5 is a DSC and TGA thermogram corresponding to crystalline Form 2. (See Example 2.)

FIG. 6 is an asymmetric unit of crystalline Form 2 from a single crystal structure. (See Example 2.)

FIG. 7 is a plasma concentration vs. time profile for crystalline Form A after a 300 mg/kg dose in NOD/SCID mice. (See Example 3.)

FIG. 8 is a plasma concentration vs. time profile for crystalline Form 2 after a 300 mg/kg dose in NOD/SCID mice. (See Example 3.)

FIG. 9 is a brain and plasma concentration vs. time profile for crystalline Form 2 after 100 and 300 mg/kg QD repeated oral dose in NOD/SCID female mice. (See Example 4.)

FIG. 10 is a brain and plasma concentration vs. time profile for crystalline Form 2 after 100 mg/kg oral dose in SD male and female rats. (See Example 5.)

FIG. 11 provides a dose escalating schema for administration of the USP1 inhibitor 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound I). (See Example 6.)

### DETAILED DESCRIPTION OF THE INVENTION

### I. Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

The characterizing data for XRPD, DSC, and TGA that are referenced throughout the application and claims are determined using the instruments and conditions specified at the beginning of the Examples section under the subheading “Instrumental Conditions.”

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

“USP1” and “ubiquitin-specific-processing protease 1” as used herein refer to any native polypeptide or USP1-encoding polynucleotide. The term “USP1” encompasses “full-length,” unprocessed USP1 polypeptide as well as any forms ofUSP1 that result from processing within the cell (e.g., removal of the signal peptide). The term also encompasses naturally occurring variants of USP1, e.g., those encoded by splice variants and allelic variants. The USP1 polypeptides described herein can be isolated from a variety of sources, such as from human tissue types or from another source, or prepared by recombinant or synthetic methods. Human USP1 sequences are known and include, for example, the sequences publicly available as UniProt No. 094782 (including isoforms). As used herein, the term “human USP1 protein” refers to LSP1 protein comprising the amino acid sequence of SEQ ID NO: 1

(SEQ ID NO: 1)
MPGVIPSESNGLSRGSPSKKNRLSLKFFQKKETKRALDFTDSQENEEKA
SEYRASEIDQVVPAAQSSPINCEKRENLLPFVGLNNLGNTCYLNSILQV
LYFCPGFKSGVKHLFNIISRKKEALKDEANQKDKGNCKEDSLASYELIC
SLQSLIISVEQLQASFLLNPEKYTDELATQPRRLLNTLRELNPMYEGYL
QHDAQEVLQCILGNIQETCQLLKKEEVKNVAELPTKVEEIPHPKEEMNG
INSIEMDSMRHSEDFKEKLPKGNGKRKSDTEFGNMKKKVKLSKEHQSLE
ENQRQTRSKRKATSDTLESPPKIIPKYISENESPRPSQKKSRVKINWLK
SATKQPSILSKFCSLGKITTNQGVKGQSKENECDPEEDLGKCESDNTTN
GCGLESPGNTVTPVNVNEVKPINKGEEQIGFELVEKLFQGQLVLRTRCL
ECESLTERREDFQDISVPVQEDELSKVEESSEISPEPKTEMKTLRWAIS
QFASVERIVGEDKYFCENCHHYTEAERSLLFDKMPEVITIHLKCFAASG
LEFDCYGGGLSKINTPLLTPLKLSLEEWSTKPTNDSYGLFAVVMHSGIT
ISSGHYTASVKVTDLNSLELDKGNFVVDQMCEIGKPEPLNEEEARGVVE
NYNDEEVSIRVGGNTQPSKVLNKKNVEAIGLLGGQKSKADYELYNKASN
PDKVASTAFAENRNSETSDTTGTHESDRNKESSDQTGINISGFENKISY
VVQSLKEYEGKWLLFDDSEVKVTEEKDFLNSLSPSTSPTSTPYLLFYKK
L.

USP1 is a deubiquitinating enzyme that acts as part of a complex with UAF1. USP1's “deubiquitinase activity” includes its ability to deubiquitinate as part of the USP1-UAF1 complex.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some aspects, by “reduce” or “inhibit” is meant the ability to cause an overall decrease of 20% or greater. In some aspects, by “reduce” or “inhibit” is meant the ability to cause an overall decrease of 50% or greater. In some aspects, by “reduce” or “inhibit” is meant the ability to cause an overall decrease of 75%, 85%, 90%, 95%, or greater. In some aspects, the amount noted above is inhibited or decreased over a period of time, relative to a control over the same period of time.

The term “USP1 inhibitor” or “inhibitor of USP1” refers to a compound capable of inhibiting one or more activities or functions of a USP1 protein. It should be appreciated that the activity or function of the one or more USP1 proteins can be inhibited in vitro or in vivo. Non-limiting examples of activities and functions of USP1 include deubiquitinase activity, and formation of a complex with UAF1. See e.g., WO2020/132269, which is herein incorporated by reference in its entirety.

The term “6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine” or “Compound I” refers to a compound having the structure below:

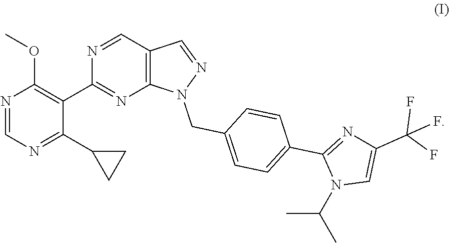 [SMILES: COc1ncnc(C2CC2)c1-c1ncc2cnn(Cc3ccc(-c4nc(C(F)(F)F)cn4C(C)C)cc3)c2n1]

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include approaches for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In the context of cancer, the terms “treat,” “treating,” and “treatment” include, but are not limited to, inhibiting growth of cancer cells, inhibiting replication of cancer cells, lessening of overall tumor burden, and delaying, halting, or slowing tumor growth, progression, or metastasis.

As used herein, the terms “cancer” and “tumor” refer to or describe the physiological condition in mammals in which a population of cells are characterized by unregulated cell growth. The terms encompass solid cancers. Examples of cancer include but are not limited to, DNA repair pathway deficient cancers and homologous recombination deficiency (HRD) cancers. Additional examples of cancer include, but are not limited to, ovarian cancer, breast cancer (including triple negative breast cancer), lung cancer (including non-small cell lung cancer (NSCLC)), and osteosarcoma. The cancer can be BRCA1 and/or BRCA2 wildtype. The cancer can also be BRCA1 (somatic and/or germline) and/or BRCA2 (somatic and/or germline) mutant. The cancer can further be a PARP inhibitor refractory or resistant cancer, or a PARP inhibitor refractory or resistant BRCA1 or BRCA2-mutant cancer.

A “therapeutically effective amount” of a substance is also one in which any toxic or detrimental effects of the substance are outweighed by the therapeutically beneficial effects. A therapeutically effective amount can be delivered in one or more administrations. A therapeutically effective amount refers to an amount effective, at dosages and for periods of time necessary, to achieve the desired therapeutic effect.

The terms “administer,” “administering,” “administration,” and the like refer to methods that can be used to enable delivery of the therapeutic agent to the desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations can be sterile.

The terms “pharmaceutically acceptable carrier,” “pharmaceutically acceptable excipient,” “physiologically acceptable carrier,” or “physiologically acceptable excipient” refer to a pharmaceutically-acceptable material, composition, or vehicle, such as a liquid or solid filler, diluent, excipient, solvent, or encapsulating material. A pharmaceutically acceptable carrier is non-toxic to recipients at the dosages and concentrations employed and is compatible with other ingredients of the formulation. The pharmaceutically acceptable carrier is appropriate for the formulation employed. In some aspects, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004 (incorporated herein by reference).

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” can be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “solvate” refers to a compound provided herein or a salt thereof, which further includes a stoichiometric or non-stoichiometric amount of solvent bound by non-covalent intermolecular forces. Where the solvent is water, the solvate is a hydrate. Where the solvent includes ethanol, the compound can be an ethanol solvate.

The term “polymorph” as used herein refers to a crystalline form of a compound or a salt, hydrate, or solvate thereof, in a particular crystal packing arrangement. All polymorphs have the same elemental composition. The term “crystalline,” as used herein, refers to a solid state form which consists of orderly arrangement of structural units. Different crystalline forms of the same compound, or a salt, co-crystal, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility. See, e.g., Remington's Pharmaceutical Sciences, 18^{th} ed., Mack Publishing, Easton PA, 173 (1990); The United States Pharmacopeia, 23^{rd} ed., 1843-1844 (1995) (incorporated herein by reference).

Crystalline forms are most commonly characterized by X-ray powder diffraction (XRPD). An XRPD pattern of reflections (peaks, typically expressed in degrees 2-theta) is commonly considered a fingerprint of a particular crystalline form. The relative intensities of the XRPD peaks can widely vary depending on, inter alia, the sample preparation technique, crystal size distribution, filters, the sample mounting procedure, and the particular instrument employed. In some aspects, new peaks may be observed or existing peaks may disappear, depending on the type of instrument or the settings. In some aspects, any particular peak in an XRPD pattern may appear as a singlet, doublet, triplet, quartet, or multiplet, depending on the type of instrument or the settings, the sensitivity of the instrument, measuring conditions, and/or purity of the crystalline form. In some aspects, any particular peak in an XRPD may appear in a symmetric shape or in an asymmetric shape, e.g., having a shoulder. A skilled artisan understanding these variations is capable of discriminating or ascertaining the defining features or characteristics of a particular crystal form using XRPD, as well as using other known physicochemical techniques.

The term “amorphous” as applied to a compound refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”).

The term “anhydrate” as applied to a compound refers to a solid state wherein the compound contains no structural water within the crystal lattice.

The term “about,” as used herein, includes the recited number ±10%. Thus, “about 10” means 9 to 11. As is understood by one skilled in the art, reference to “about” a value or parameter herein includes (and describes) aspects that are directed to that value or parameter per se. For example, description referring to “about X” includes description of “X.”

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

### II. Methods of Treating Cancer with USP1 Inhibitors

Provided herein are methods of treating cancers, e.g., solid tumors, with a USP1 inhibitor (e.g., Compound I or a pharmaceutically acceptable salt, solvate, hydrate, or co-crystal thereof).

In some aspects, the methods comprise administering about 75 mg to about 1000 mg of the USP1 inhibitor (e.g., Compound I) or administering an amount of a salt, solvate, hydrate, or co-crystal thereof equivalent to about 75 mg to about 100 mg of the USP1 inhibitor. In some aspects, the methods comprise administering about 75 mg of the USP1 inhibitor (e.g., Compound I) or administering an amount of a salt, solvate, hydrate, or co-crystal thereof equivalent to about 75 mg of the USP1 inhibitor. In some aspects, the methods comprise administering about 100 mg of the USP1 inhibitor (e.g., Compound I) or administering an amount of a salt, solvate, hydrate, or co-crystal thereof equivalent to about 100 mg of the USP1 inhibitor. In some aspects, the methods comprise administering about 150 mg of the USP1 inhibitor (e.g., Compound I)) or administering an amount of a salt, solvate, hydrate, or co-crystal thereof equivalent to about 150 mg of the USP1 inhibitor. In some aspects, the methods comprise administering about 200 mg of the USP1 inhibitor (e.g., Compound 1)) or administering an amount of a salt, solvate, hydrate, or co-crystal thereof equivalent to about 200 mg of the USP1 inhibitor. In some aspects, the methods comprise administering about 250 mg of the USP1 inhibitor (e.g., Compound I) or administering an amount of a salt, solvate, hydrate, or co-crystal thereof equivalent to about 250 mg of the USP1 inhibitor. In some aspects, the methods comprise administering about 300 mg of the USP1 inhibitor (e.g., Compound I) or administering an amount of a salt, solvate, hydrate, or co-crystal thereof equivalent to about 300 mg of the USP1 inhibitor. In some aspects, the methods comprise administering about 350 mg of the USP1 inhibitor (e.g., Compound I) or administering an amount of a salt, solvate, hydrate, or co-crystal thereof equivalent to about 350 mg of the USP1 inhibitor. In some aspects, the methods comprise administering about 400 mg of the USP1 inhibitor (e.g., Compound I) or administering an amount of a salt, solvate, hydrate, or co-crystal thereof equivalent to about 450 mg of the USP1 inhibitor. In some aspects, the methods comprise administering about 450 mg of the USP1 inhibitor (e.g., Compound I) or administering an amount of a salt, solvate, hydrate, or co-crystal thereof equivalent to about 400 mg of the USP1 inhibitor. In some aspects, the methods comprise administering about 500 mg of the USP1 inhibitor (e.g., Compound 1) or administering an amount of a salt, solvate, hydrate, or co-crystal thereof equivalent to about 500 mg of the USP1 inhibitor. In some aspects, the methods comprise administering about 550 mg of the USP1 inhibitor (e.g., Compound I) or administering an amount of a salt, solvate, hydrate, or co-crystal thereof equivalent to about 550 mg of the USP1 inhibitor. In some aspects, the methods comprise administering about 600 mg of the USP1 inhibitor (e.g., Compound I) or administering an amount of a salt, solvate, hydrate, or co-crystal thereof equivalent to about 600 mg of the USP1 inhibitor. In some aspects, the methods comprise administering about 650 mg of the USP1 inhibitor (e.g., Compound I) or administering an amount of a salt, solvate, hydrate, or co-crystal thereof equivalent to about 650 mg of the USP1 inhibitor. In some aspects, the methods comprise administering about 700 mg of the USP1 inhibitor (e.g., Compound I) or administering an amount of a salt, solvate, hydrate, or co-crystal thereof equivalent to about 700 mg of the USP1 inhibitor. In some aspects, the methods comprise administering about 750 mg of the USP1 inhibitor (e.g., Compound I) or administering an amount of a salt, solvate, hydrate, or co-crystal thereof equivalent to about 750 mg of the USP1 inhibitor. In some aspects, the methods comprise administering about 800 mg of the USP1 inhibitor (e.g., Compound I) or administering an amount of a salt, solvate, hydrate, or co-crystal thereof equivalent to about 800 mg of the USP1 inhibitor. In some aspects, the methods comprise administering about 850 mg of the USP1 inhibitor (e.g., Compound I) or administering an amount of a salt, solvate, hydrate, or co-crystal thereof equivalent to about 850 mg of the USP1 inhibitor. In some aspects, the methods comprise administering about 900 mg of the USP1 inhibitor (e.g., Compound I) or administering an amount of a salt, solvate, hydrate, or co-crystal thereof equivalent to about 900 mg of the USP1 inhibitor. In some aspects, the methods comprise administering about 950 mg of the USP1 inhibitor (e.g., Compound I) or administering an amount of a salt, solvate, hydrate, or co-crystal thereof equivalent to about 950 mg of the USP1 inhibitor. In some aspects, the methods comprise administering about 1000 mg of the USP1 inhibitor (e.g., Compound I) or administering an amount of a salt, solvate, hydrate, or co-crystal thereof equivalent to about 1000 mg of the USP1 inhibitor. In some aspects, the methods comprise administering about 1050 mg, or about 1100 mg, or about 1150 mg, or about 1200 mg, or about 1250 mg, or about 1300 mg, or about 1350 mg, or about 1400 mg, or about 1450 mg, or about 1500 mg, or about 1550 mg, or about 1600 mg, or about 1650 mg, or about 1700 mg, or about 1750 mg, or about 1800 mg, or about 1850 mg, or about 1900 mg, or about 1950 mg, or about 2000 mg, or about 2100 mg or about 2200 mg, or about 2300 mg, or about 2400 mg, or about 2500 mg of the USP1 inhibitor (e.g., Compound 1) or administering an amount of a salt, solvate, hydrate, or co-crystal thereof equivalent to about 1050 mg, or about 1100 mg, or about 1150 mg, or about 1200 mg, or about 1250 mg, or about 1300 mg, or about 1350 mg, or about 1400 mg, or about 1450 mg, or about 1500 mg, or about 1550 mg, or about 1600 mg, or about 1650 mg, or about 1700 mg, or about 1750 mg, or about 1800 mg, or about 1850 mg, or about 1900 mg, or about 1950 mg, or about 2000 mg, or about 2100 mg or about 2200 mg, or about 2300 mg, or about 2400 mg, or about 2500 mg of the USP1 inhibitor of the USP1 inhibitor.

In some aspects, the methods comprise administering about 50 mg to about 2500 mg, or about 50 mg to about 2000 mg, or about 50 mg to about 1500 mg, or about 100 mg to about 1000 mg, or about 200 mg to about 800 mg of a USP1 inhibitor (e.g., Compound I) or administering an amount of a salt, solvate, hydrate, or co-crystal thereof equivalent to about 50 mg to about 2500 mg, or about 50 mg to about 2000 mg, or about 50 mg to about 1500 mg, or about 100 mg to about 1000 mg, or about 200 mg to about 800 mg of the USP1 inhibitor.

In some aspects, the methods comprise administering about 75 mg to about 1000 mg of the USP1 inhibitor (e.g., Compound I or a pharmaceutically acceptable salt, solvate, hydrate, or co-crystal thereof). In some aspects, the methods comprise administering about 75 mg of the USP1 inhibitor (e.g., Compound I or a pharmaceutically acceptable salt, solvate, hydrate, or co-crystal thereof). In some aspects, the methods comprise administering about 100 mg of the USP1 inhibitor (e.g., Compound I or a pharmaceutically acceptable salt, solvate, hydrate, or co-crystal thereof). In some aspects, the methods comprise administering about 150 mg of the USP1 inhibitor (e.g., Compound I or a pharmaceutically acceptable salt, solvate, hydrate, or co-crystal thereof). In some aspects, the methods comprise administering about 200 mg of the USP1 inhibitor (e.g., Compound I or a pharmaceutically acceptable salt, solvate, hydrate, or co-crystal thereof). In some aspects, the methods comprise administering about 250 mg of the USP1 inhibitor (e.g., Compound I or a pharmaceutically acceptable salt, solvate, hydrate, or co-crystal thereof). In some aspects, the methods comprise administering about 300 mg of the USP1 inhibitor (e.g., Compound I or a pharmaceutically acceptable salt, solvate, hydrate, or co-crystal thereof. In some aspects, the methods comprise administering about 350 mg of the USP1 inhibitor (e.g., Compound I or a pharmaceutically acceptable salt, solvate, hydrate, or co-crystal thereof). In some aspects, the methods comprise administering about 400 mg of the USP1 inhibitor (e.g., Compound I or a pharmaceutically acceptable salt, solvate, hydrate, or co-crystal thereof). In some aspects, the methods comprise administering about 500 mg of the USP1 inhibitor (e.g., Compound I or a pharmaceutically acceptable salt, solvate, hydrate, or co-crystal thereof). In some aspects, the methods comprise administering about 550 mg of the USP1 inhibitor (e.g., Compound I or a pharmaceutically acceptable salt, solvate, hydrate, or co-crystal thereof). In some aspects, the methods comprise administering about 600 mg of the USP1 inhibitor (e.g., Compound I or a pharmaceutically acceptable salt, solvate, hydrate, or co-crystal thereof). In some aspects, the methods comprise administering about 650 mg of the USP1 inhibitor (e.g., Compound I or a pharmaceutically acceptable salt, solvate, hydrate, or co-crystal thereof). In some aspects, the methods comprise administering about 700 mg of the USP1 inhibitor (e.g., Compound I or a pharmaceutically acceptable salt, solvate, hydrate, or co-crystal thereof). In some aspects, the methods comprise administering about 750 mg of the USP1 inhibitor (e.g., Compound I or a pharmaceutically acceptable salt, solvate, hydrate, or co-crystal thereof). In some aspects, the methods comprise administering about 800 mg of the USP1 inhibitor (e.g., Compound I or a pharmaceutically acceptable salt, solvate, hydrate, or co-crystal thereof). In some aspects, the methods comprise administering about 850 mg of the USP1 inhibitor (e.g., Compound I or a pharmaceutically acceptable salt, solvate, hydrate, or co-crystal thereof). In some aspects, the methods comprise administering about 900 mg of the USP1 inhibitor (e.g., Compound I or a pharmaceutically acceptable salt, solvate, hydrate, or co-crystal thereof). In some aspects, the methods comprise administering about 950 mg of the USP1 inhibitor (e.g., Compound I or a pharmaceutically acceptable salt, solvate, hydrate, or co-crystal thereof). In some aspects, the methods comprise administering about 1000 mg of the USP1 inhibitor (e.g., Compound I or a pharmaceutically acceptable salt, solvate, hydrate, or co-crystal thereof). In some aspects, the methods comprise administering about 1050 mg, or about 1100 mg, or about 1150 mg, or about 1200 mg, or about 1250 mg, or about 1300 mg, or about 1350 mg, or about 1400 mg, or about 1450 mg, or about 1500 mg, or about 1550 mg, or about 1600 mg, or about 1650 mg, or about 1700 mg, or about 1750 mg, or about 1800 mg, or about 1850 mg, or about 1900 mg, or about 1950 mg, or about 2000 mg, or about 2100 mg or about 2200 mg, or about 2300 mg, or about 2400 mg, or about 2500 mg of the USP1 inhibitor (e.g., Compound I or a pharmaceutically acceptable salt, solvate, hydrate, or co-crystal thereof).

In some aspects, the methods comprise administering about 50 mg to about 2500 mg, or about 50 mg to about 2000 mg, or about 50 mg to about 1500 mg, or about 100 mg to about 1000 mg, or about 200 mg to about 800 mg of the USP1 inhibitor (e.g., Compound I or a pharmaceutically acceptable salt, solvate, hydrate, or co-crystal thereof).

In some aspects, the administration is oral. The methods can comprise administering about the USP1 inhibitor about once daily. In some aspects described in the paragraph above, the USP1 inhibitor is administered as a divided dose over the course of a day. For example, in some aspects, a the USP1 inhibitor is administered over a course of two administrations in a day (e.g., if the dose is 1000 mg or equivalents thereof, the USP1 inhibitor is administered in a first dose of 500 mg and a second dose of 500 mg on the same day, wherein the first dose and the second dose are not simultaneous). The amount of the first dose and the second dose may be the same or different.

The methods can comprise administering the USP1 inhibitor at least twice (e.g., on two consecutive days). The methods can comprise administering the USP1 inhibitor at least three times (e.g., on three consecutive days). The methods can comprise administering the USP1 inhibitor at least four times (e.g., on four consecutive days). The methods can comprise administering the USP1 inhibitor at least five times (e.g., on five consecutive days). The methods can comprise administering the USP1 inhibitor about 10 times (e.g., on about 10 consecutive days). The methods can comprise administering the USP1 inhibitor about 15 times (e.g., on about 15 consecutive days). The methods can comprise administering the USP1 inhibitor about 20 times (e.g., on about 20 consecutive days). The methods can comprise administering the USP1 inhibitor about 25 times (e.g., on about 25 consecutive days). The methods can comprise administering the USP1 inhibitor about 28 times (e.g., on about 28 consecutive days).

The methods can comprise administering the USP1 inhibitor for at least one month, or at least 2 months, or at least 3 months, or at least 4 months, or at least 5 months, or at least 6 months, or at least 7 months, or at least 8 months, or at least 9 months, or at least 10 months, or at least 11 months, or at least 1 year, or at least 18 months, or at least 2 years, or more.

The methods can comprise administering about the USP1 inhibitor about once every 21 days (about once every three weeks). The methods can comprise administering the USP1 inhibitor at least twice (e.g., about 21 days apart). The methods can comprise administering the USP1 inhibitor at least three times (e.g., about 21 days apart). The methods can comprise administering the USP1 inhibitor at least four times (e.g., about 21 days apart). The methods can comprise administering the USP1 inhibitor at least five times (e.g., about 21 days apart).

Also provided herein are uses of USP1 inhibitors (e.g., Compound I or a pharmaceutically acceptable salt, solvate, hydrate, or co-crystal thereof) in a medicament for treating a solid tumor. In some aspects, the medicament is formulated for oral administration.

In some aspects, such a medicament comprises about 75 mg to about 1000 mg of a USP1 inhibitor (e.g., Compound I) or an amount of a pharmaceutically acceptable salt, solvate, hydrate, or co-crystal thereof equivalent to about 75 mg to about 1000 mg of the USP1 inhibitor. In some aspects, such a medicament comprises about 75 mg of a USP1 inhibitor (e.g., Compound I) or an amount of a pharmaceutically acceptable salt, solvate, hydrate, or co-crystal thereof equivalent to about 75 mg the USP1 inhibitor. In some aspects, such a medicament comprises about 100 mg of a USP1 inhibitor (e.g., Compound I) or an amount of a pharmaceutically acceptable salt, solvate, hydrate, or co-crystal thereof equivalent to about 100 mg the USP1 inhibitor. In some aspects, such a medicament comprises about 150 mg of a USP1 inhibitor (e.g., Compound I) or an amount of a pharmaceutically acceptable salt, solvate, hydrate, or co-crystal thereof equivalent to about 150 mg the USP1 inhibitor. In some aspects, such a medicament comprises about 200 mg of a USP1 inhibitor (e.g., Compound I) or an amount of a pharmaceutically acceptable salt, solvate, hydrate, or co-crystal thereof equivalent to about 200 mg the USP1 inhibitor. In some aspects, such a medicament comprises about 250 mg of a USP1 inhibitor (e.g., Compound I) or an amount of a pharmaceutically acceptable salt, solvate, hydrate, or co-crystal thereof equivalent to about 250 mg the USP1 inhibitor. In some aspects, such a medicament comprises about 300 mg of a USP1 inhibitor (e.g., Compound I) or an amount of a pharmaceutically acceptable salt, solvate, hydrate, or co-crystal thereof equivalent to about 300 mg the USP1 inhibitor. In some aspects, such a medicament comprises about 350 mg of a USP1 inhibitor (e.g., Compound I) or an amount of a pharmaceutically acceptable salt, solvate, hydrate, or co-crystal thereof equivalent to about 350 mg the USP1 inhibitor. In some aspects, such a medicament comprises about 400 mg of a USP1 inhibitor (e.g., Compound I) or an amount of a pharmaceutically acceptable salt, solvate, hydrate, or co-crystal thereof equivalent to about 400 mg the USP1 inhibitor. In some aspects, such a medicament comprises about 450 mg of a USP1 inhibitor (e.g., Compound I) or an amount of a pharmaceutically acceptable salt, solvate, hydrate, or co-crystal thereof equivalent to about 450 mg the USP1 inhibitor. In some aspects, such a medicament comprises about 500 mg of a USP1 inhibitor (e.g., Compound I) or an amount of a pharmaceutically acceptable salt, solvate, hydrate, or co-crystal thereof equivalent to about 500 mg the USP1 inhibitor. In some aspects, such a medicament comprises about 550 mg of a USP1 inhibitor (e.g., Compound I) or an amount of a pharmaceutically acceptable salt, solvate, hydrate, or co-crystal thereof equivalent to about 550 mg the USP1 inhibitor. In some aspects, such a medicament comprises about 600 mg of a USP1 inhibitor (e.g., Compound I) or an amount of a pharmaceutically acceptable salt, solvate, hydrate, or co-crystal thereof equivalent to about 600 mg the USP1 inhibitor. In some aspects, such a medicament comprises about 650 mg of a USP1 inhibitor (e.g., Compound I) or an amount of a pharmaceutically acceptable salt, solvate, hydrate, or co-crystal thereof equivalent to about 650 mg the USP1 inhibitor. In some aspects, such a medicament comprises about 700 mg of a USP1 inhibitor (e.g., Compound I) or an amount of a pharmaceutically acceptable salt, solvate, hydrate, or co-crystal thereof equivalent to about 700 mg the USP1 inhibitor. In some aspects, such a medicament comprises about 750 mg of a USP1 inhibitor (e.g., Compound I) or an amount of a pharmaceutically acceptable salt, solvate, hydrate, or co-crystal thereof equivalent to about 750 mg the USP1 inhibitor. In some aspects, such a medicament comprises about 800 mg of a USP1 inhibitor (e.g., Compound I) or an amount of a pharmaceutically acceptable salt, solvate, hydrate, or co-crystal thereof equivalent to about 800 mg the USP1 inhibitor. In some aspects, such a medicament comprises about 850 mg of a USP1 inhibitor (e.g., Compound I) or an amount of a pharmaceutically acceptable salt, solvate, hydrate, or co-crystal thereof equivalent to about 850 mg the USP1 inhibitor. In some aspects, such a medicament comprises about 900 mg of a USP1 inhibitor (e.g., Compound I) or an amount of a pharmaceutically acceptable salt, solvate, hydrate, or co-crystal thereof equivalent to about 900 mg the USP1 inhibitor. In some aspects, such a medicament comprises about 950 mg of a USP1 inhibitor (e.g., Compound I) or an amount of a pharmaceutically acceptable salt, solvate, hydrate, or co-crystal thereof equivalent to about 950 mg the USP1 inhibitor. In some aspects, such a medicament comprises about 1000 mg of a USP1 inhibitor (e.g., Compound I) or an amount of a pharmaceutically acceptable salt, solvate, hydrate, or co-crystal thereof equivalent to about 1000 mg the USP1 inhibitor. In some aspects, such a medicament comprises about 1050 mg, or about 1100 mg, or about 1150 rmg, or about 1200 mg, or about 1250 mg, or about 1300 mg, or about 1350 mg, or about 1400 mg, or about 1450 mg, or about 1500 mg, or about 1550 mg, or about 1600 mg, or about 1650 mg, or about 1700 mg, or about 1750 mg, or about 1800 mg, or about 1850 mg, or about 1900 mg, or about 1950 mg, or about 2000 mg, or about 2100 mg or about 2200 mg, or about 2300 mg, or about 2400 mg, or about 2500 mg of a USP1 inhibitor (e.g., Compound I) or an amount of a pharmaceutically acceptable salt, solvate, hydrate, or co-crystal thereof equivalent to about 1050 mg, or about 1100 mg, or about 1150 mg, or about 1200 mg, or about 1250 mg, or about 1300 mg, or about 1350 mg, or about 1400 mg, or about 1450 mg, or about 1500 mg, or about 1550 mg, or about 1600 mg, or about 1650 mg, or about 1700 mg, or about 1750 mg, or about 1800 mg, or about 1850 mg, or about 1900 mg, or about 1950 mg, or about 2000 mg, or about 2100 mg or about 2200 mg, or about 2300 mg, or about 2400 mg, or about 2500 mg of the USP1 inhibitor. In some aspects, such a medicament comprises about 50 ng to about 2500 mg, or about 50 mg to about 2000 mg, or about 50 mg to about 1500 mg, or about 100 mg to about 1000 mg, or about 200 mg to about 800 mg of a USP1 inhibitor (e.g., Compound I) or an amount of a pharmaceutically acceptable salt, solvate, hydrate, or co-crystal thereof equivalent to about 50 mg to about 2500 mg, or about 50 mg to about 2000 mg, or about 50 mg to about 1500 mg, or about 100 mg to about 1000 mg, or about 200 mg to about 800 mg of the USP1 inhibitor.

In some aspects, such a medicament comprises about 75 mg to about 1000 mg of a USP1 inhibitor (e.g., Compound I or a pharmaceutically acceptable salt, solvate, hydrate, or co-crystal thereof). In some aspects, such a medicament comprises about 75 mg of a USP1 inhibitor (e.g., Compound I or a pharmaceutically acceptable salt, solvate, hydrate, or co-crystal thereof). In some aspects, such a medicament comprises about 100 mg of a USP1 inhibitor (e.g., Compound I or a pharmaceutically acceptable salt, solvate, hydrate, or co-crystal thereof). In some aspects, such a medicament comprises about 150 mg of a USP1 inhibitor (e.g., Compound I or a pharmaceutically acceptable salt, solvate, hydrate, or co-crystal thereof). In some aspects, such a medicament comprises about 200 mg of a USP1 inhibitor (e.g., Compound I or a pharmaceutically acceptable salt, solvate, hydrate, or co-crystal thereof). In some aspects, such a medicament comprises about 250 mg of a USP1 inhibitor (e.g., Compound I or a pharmaceutically acceptable salt, solvate, hydrate, or co-crystal thereof). In some aspects, such a medicament comprises about 300 mg of a USP1 inhibitor (e.g., Compound I or a pharmaceutically acceptable salt, solvate, hydrate, or co-crystal thereof). In some aspects, such a medicament comprises about 350 mg of a USP1 inhibitor (e.g., Compound I or a pharmaceutically acceptable salt, solvate, hydrate, or co-crystal thereof). In some aspects, such a medicament comprises about 400 mg of a USP1 inhibitor (e.g., Compound I or a pharmaceutically acceptable salt, solvate, hydrate, or co-crystal thereof). In some aspects, such a medicament comprises about 450 mg of a USP1 inhibitor (e.g., Compound I or a pharmaceutically acceptable salt, solvate, hydrate, or co-crystal thereof). In some aspects, such a medicament comprises about 500 mg of a USP1 inhibitor (e.g., Compound I or a pharmaceutically acceptable salt, solvate, hydrate, or co-crystal thereof). In some aspects, such a medicament comprises about 550 mg of a USP1 inhibitor (e.g., Compound I or a pharmaceutically acceptable salt, solvate, hydrate, or co-crystal thereof). In some aspects, such a medicament comprises about 600 mg of a USP1 inhibitor (e.g., Compound I or a pharmaceutically acceptable salt, solvate, hydrate, or co-crystal thereof). In some aspects, such a medicament comprises about 650 mg of a USP1 inhibitor (e.g., Compound I or a pharmaceutically acceptable salt, solvate, hydrate, or co-crystal thereof). In some aspects, such a medicament comprises about 700 mg of a USP1 inhibitor (e.g., Compound I or a pharmaceutically acceptable salt, solvate, hydrate, or co-crystal thereof). In some aspects, such a medicament comprises about 750 mg of a USP1 inhibitor (e.g., Compound I or a pharmaceutically acceptable salt, solvate, hydrate, or co-crystal thereof). In some aspects, such a medicament comprises about 800 mg of a USP1 inhibitor (e.g., Compound I or a pharmaceutically acceptable salt, solvate, hydrate, or co-crystal thereof). In some aspects, such a medicament comprises about 850 mg of a USP1 inhibitor (e.g., Compound I or a pharmaceutically acceptable salt, solvate, hydrate, or co-crystal thereof). In some aspects, such a medicament comprises about 900 mg of a USP1 inhibitor (e.g., Compound I or a pharmaceutically acceptable salt, solvate, hydrate, or co-crystal thereof). In some aspects, such a medicament comprises about 950 mg of a USP1 inhibitor (e.g., Compound I or a pharmaceutically acceptable salt, solvate, hydrate, or co-crystal thereof). In some aspects, such a medicament comprises about 1000 mg of a USP1 inhibitor (e.g., Compound I or a pharmaceutically acceptable salt, solvate, hydrate, or co-crystal thereof). In some aspects, such a medicament comprises about 1050 mg, or about 1100 mg, or about 1150 mg, or about 1200 mg, or about 1250 mg, or about 1300 mg, or about 1350 mg, or about 1400 mg, or about 1450 mg, or about 1500 mg, or about 1550 mg, or about 1600 mg, or about 1650 mg, or about 1700 mg, or about 1750 mg, or about 1800 mg, or about 1850 mg, or about 1900 mg, or about 1950 mg, or about 2000 mg, or about 2100 mg or about 2200 mg, or about 2300 mg, or about 2400 mg, or about 2500 mg of a USP1 inhibitor (e.g., Compound I or a pharmaceutically acceptable salt, solvate, hydrate, or co-crystal thereof). In some aspects, such a medicament comprises about 50 mg to about 2500 mg, or about 50 mg to about 2000 mg, or about 50 mg to about 1500 mg, or about 100 mg to about 1000 mg, or about 200 mg to about 800 mg of a USP1 inhibitor (e.g., Compound I or a pharmaceutically acceptable salt, solvate, hydrate, or co-crystal thereof).

In some aspects, such a medicament is for administration about once per day. In some aspects, such a medicament is for administration at least twice (e.g., one two consecutive days). In some aspects, such a medicament is for administration at least three times (e.g., on three consecutive days). In some aspects, such a medicament is for administration at least four times (e.g., on four consecutive days). In some aspects, such a medicament is for administration at least five times (e.g., on five consecutive days). In some aspects, such a medicament is for administration at least five times (e.g., on five consecutive days). In some aspects, such a medicament is for administration about 28 times (e.g., on about 28 consecutive days). In some aspects, such a medicament is for administration at least three times, at least 4 times, or at least 5 times, or at least times, or at least 15 times, or at least 20 days, or at least 25 days, or at least 30 days, or at least 60 days, or at least 90 days, or at least 120 days, or least 150 days, or at least 180 days, or at least 210 days, or at least 240 days, or at least 270 days, or at least 300 days, or at least 330 days, or at least 360 days, or at least 365 days, or more. In some aspects, such a medicament is for administration for a period of at least 3 days, or at least 4 days, or at least 5 days, or at least 10 days, or at least 15 days, or at least 20 days, or at least 25 days, or at least 1 month, or at least 2 months, or at least 3 months, or at least 4 months, or at least 5 months, or at least 6 months, or at least 7 months, or at least 8 months, or at least 9 months, or at least 10 months, or at least 11 months, or at least 1 year, or at least 18 months, or at least 2 years, or more.

In some aspects, such a medicament is for administration about once every 21 days (about once every three weeks. In some aspects, such a medicament is for administration at least twice (e.g., about 21 days apart). In some aspects, such a medicament is for administration at least three times (e.g., about 21 days apart). In some aspects, such a medicament is for administration at least four times (e.g., about 21 days apart). In some aspects, such a medicament is for administration at least five times (e.g., about 21 days apart).

In some aspects, such a medicament is formulated for administration of about 75 mg to about 1000 mg of a USP1 inhibitor (e.g., Compound I or a pharmaceutically acceptable salt, solvate, hydrate, or co-crystal thereof). In some aspects, such a medicament is formulated for administration of about 75 mg of a USP1 inhibitor (e.g., Compound I or a pharmaceutically acceptable salt, solvate, hydrate, or co-crystal thereof). In some aspects, such a medicament is formulated for administration of about 100 mg of a USP1 inhibitor (e.g., Compound I or a pharmaceutically acceptable salt, solvate, hydrate, or co-crystal thereof). In some aspects, such a medicament is formulated for administration of about 150 mg of a USP1 inhibitor (e.g., Compound I or a pharmaceutically acceptable salt, solvate, hydrate, or co-crystal thereof). In some aspects, such a medicament is formulated for administration of about 200 mg of a USP1 inhibitor (e.g., Compound I or a pharmaceutically acceptable salt, solvate, hydrate, or co-crystal thereof). In some aspects, such a medicament is formulated for administration of about 250 mg of a USP1 inhibitor (e.g., Compound I or a pharmaceutically acceptable salt, solvate, hydrate, or co-crystal thereof). In some aspects, such a medicament is formulated for administration of about 300 mg of a USP1 inhibitor (e.g., Compound I or a pharmaceutically acceptable salt, solvate, hydrate, or co-crystal thereof). In some aspects, such a medicament is formulated for administration of about 350 mg of a USP1 inhibitor (e.g., Compound I or a pharmaceutically acceptable salt, solvate, hydrate, or co-crystal thereof). In some aspects, such a medicament is formulated for administration of about 400 mg of a USP1 inhibitor (e.g., Compound I or a pharmaceutically acceptable salt, solvate, hydrate, or co-crystal thereof). In some aspects, such a medicament is formulated for administration of about 450 mg of a USP1 inhibitor (e.g., Compound I or a pharmaceutically acceptable salt, solvate, hydrate, or co-crystal thereof). In some aspects, such a medicament is formulated for administration of about 500 mg of a USP1 inhibitor (e.g., Compound I or a pharmaceutically acceptable salt, solvate, hydrate, or co-crystal thereof). In some aspects, such a medicament is formulated for administration of about 550 mg of a USP1 inhibitor (e.g., Compound I or a pharmaceutically acceptable salt, solvate, hydrate, or co-crystal thereof). In some aspects, such a medicament is formulated for administration of about 600 mg of a USP1 inhibitor (e.g., Compound I or a pharmaceutically acceptable salt, solvate, hydrate, or co-crystal thereof). In some aspects, such a medicament is formulated for administration of about 650 mg of a USP1 inhibitor (e.g., Compound I or a pharmaceutically acceptable salt, solvate, hydrate, or co-crystal thereof). In some aspects, such a medicament is formulated for administration of about 700 mg of a USP1 inhibitor (e.g., Compound I or a pharmaceutically acceptable salt, solvate, hydrate, or co-crystal thereof). In some aspects, such a medicament is formulated for administration of about 750 mg of a USP1 inhibitor (e.g., Compound I or a pharmaceutically acceptable salt, solvate, hydrate, or co-crystal thereof). In some aspects, such a medicament is formulated for administration of about 800 mg of a USP1 inhibitor (e.g., Compound I or a pharmaceutically acceptable salt, solvate, hydrate, or co-crystal thereof). In some aspects, such a medicament is formulated for administration of about 850 mg of a USP1 inhibitor (e.g., Compound I or a pharmaceutically acceptable salt, solvate, hydrate, or co-crystal thereof). In some aspects, such a medicament is formulated for administration of about 900 mg of a USP1 inhibitor (e.g., Compound I or a pharmaceutically acceptable salt, solvate, hydrate, or co-crystal thereof). In some aspects, such a medicament is formulated for administration of about 950 mg of a USP1 inhibitor (e.g., Compound I or a pharmaceutically acceptable salt, solvate, hydrate, or co-crystal thereof). In some aspects, such a medicament is formulated for administration of about 1000 mg of a USP1 inhibitor (e.g., Compound I or a pharmaceutically acceptable salt, solvate, hydrate, or co-crystal thereof). In some aspects, such a medicament is formulated for administration of about 1050 mg, or about 1100 mg, or about 1150 mg, or about 1200 mg, or about 1250 mg, or about 1300 mg, or about 1350 mg, or about 1400 mg, or about 1450 mg, or about 1500 mg, or about 1550 mg, or about 1600 mg, or about 1650 mg, or about 1700 mg, or about 1750 mg, or about 1800 mg, or about 1850 mg, or about 1900 mg, or about 1950 mg, or about 2000 mg, or about 2100 mg or about 2200 mg, or about 2300 mg, or about 2400 mg, or about 2500 mg of a USP1 inhibitor (e.g., Compound I or a pharmaceutically acceptable salt, solvate, hydrate, or co-crystal thereof). In some aspects, such a medicament is formulated for administration of about 50 mg to about 2500 mg, or about 50 mg to about 2000 mg, or about 50 mg to about 1500 mg, or about 100 mg to about 1000 mg, or about 200 mg to about 800 mg of a USP1 inhibitor (e.g., Compound I or a pharmaceutically acceptable salt, solvate, hydrate, or co-crystal thereof).

In some aspects, a medicament is formulated for administration of about 75 mg of the USP1 inhibitor or an amount of the salt, solvate, hydrate, or co-crystal thereof equivalent to about 75 mg of the USP1 inhibitor. In some aspects, a medicament is formulated for administration of about 100 mg of the USP1 inhibitor or an amount of the salt, solvate, hydrate, or co-crystal thereof equivalent to about 100 mg of the USP1 inhibitor. In some aspects, a medicament is formulated for administration of about 150 mg of the USP1 inhibitor or an amount of the salt, solvate, hydrate, or co-crystal thereof equivalent to about 150 mg of the USP1 inhibitor. In some aspects, a medicament is formulated for administration of about 200 mg of the USP1 inhibitor or an amount of the salt, solvate, hydrate, or co-crystal thereof equivalent to about 200 mg of the USP1 inhibitor. In some aspects, a medicament is formulated for administration of about 250 mg of the USP1 inhibitor or an amount of the salt, solvate, hydrate, or co-crystal thereof equivalent to about 250 mg of the USP1 inhibitor. In some aspects, a medicament is formulated for administration of about 300 mg of the USP1 inhibitor or an amount of the salt, solvate, hydrate, or co-crystal thereof equivalent to about 300 mg of the USP1 inhibitor. In some aspects, a medicament is formulated for administration of about 350 mg of the USP1 inhibitor or an amount of the salt, solvate, hydrate, or co-crystal thereof equivalent to about 350 mg of the USP1 inhibitor. In some aspects, a medicament is formulated for administration of about 400 mg of the USP1 inhibitor or an amount of the salt, solvate, hydrate, or co-crystal thereof equivalent to about 400 mg of the USP1 inhibitor. In some aspects, a medicament is formulated for administration of about 450 mg of the USP1 inhibitor or an amount of the salt, solvate, hydrate, or co-crystal thereof equivalent to about 450 mg of the USP1 inhibitor. In some aspects, a medicament is formulated for administration of about 500 mg of the USP1 inhibitor or an amount of the salt, solvate, hydrate, or co-crystal thereof equivalent to about 500 mg of the USP1 inhibitor. In some aspects, a medicament is formulated for administration of about 550 mg of the USP1 inhibitor or an amount of the salt, solvate, hydrate, or co-crystal thereof equivalent to about 550 mg of the USP1 inhibitor. In some aspects, a medicament is formulated for administration of about 600 mg of the USP1 inhibitor or an amount of the salt, solvate, hydrate, or co-crystal thereof equivalent to about 600 mg of the USP1 inhibitor. In some aspects, a medicament is formulated for administration of about 650 mg of the USP1 inhibitor or an amount of the salt, solvate, hydrate, or co-crystal thereof equivalent to about 650 mg of the USP1 inhibitor. In some aspects, a medicament is formulated for administration of about 700 mg of the USP1 inhibitor or an amount of the salt, solvate, hydrate, or co-crystal thereof equivalent to about 700 mg of the USP1 inhibitor. In some aspects, a medicament is formulated for administration of about 750 mg of the USP1 inhibitor or an amount of the salt, solvate, hydrate, or co-crystal thereof equivalent to about 750 mg of the USP1 inhibitor. In some aspects, a medicament is formulated for administration of about 800 mg of the USP1 inhibitor or an amount of the salt, solvate, hydrate, or co-crystal thereof equivalent to about 800 mg of the USP1 inhibitor. In some aspects, a medicament is formulated for administration of about 850 mg of the USP1 inhibitor or an amount of the salt, solvate, hydrate, or co-crystal thereof equivalent to about 850 mg of the USP1 inhibitor. In some aspects, a medicament is formulated for administration of about 900 mg of the USP1 inhibitor or an amount of the salt, solvate, hydrate, or co-crystal thereof equivalent to about 900 mg of the USP1 inhibitor. In some aspects, a medicament is formulated for administration of about 950 mg of the USP1 inhibitor or an amount of the salt, solvate, hydrate, or co-crystal thereof equivalent to about 950 mg of the USP1 inhibitor. In some aspects, a medicament is formulated for administration of about 1000 mg of the USP1 inhibitor or an amount of the salt, solvate, hydrate, or co-crystal thereof equivalent to about 1000 mg of the USP1 inhibitor. In some aspects, a medicament is formulated for administration of about 1050 mg of the USP1 inhibitor or an amount of the salt, solvate, hydrate, or co-crystal thereof equivalent to about 1050 mg of the USP1 inhibitor. In some aspects, a medicament is formulated for administration of about 1100 mg of the USP1 inhibitor or an amount of the salt, solvate, hydrate, or co-crystal thereof equivalent to about 1100 mg of the USP1 inhibitor. In some aspects, a medicament is formulated for administration of about 1150 mg of the USP1 inhibitor or an amount of the salt, solvate, hydrate, or co-crystal thereof equivalent to about 1150 mg of the USP1 inhibitor. In some aspects, a medicament is formulated for administration of about 1200 mg of the USP1 inhibitor or an amount of the salt, solvate, hydrate, or co-crystal thereof equivalent to about 1200 mg of the USP1 inhibitor. In some aspects, a medicament is formulated for administration of about 1250 mg of the USP1 inhibitor or an amount of the salt, solvate, hydrate, or co-crystal thereof equivalent to about 1250 mg of the USP1 inhibitor. In some aspects, a medicament is formulated for administration of about 1300 mg of the USP1 inhibitor or an amount of the salt, solvate, hydrate, or co-crystal thereof equivalent to about 1300 mg of the USP1 inhibitor. In some aspects, a medicament is formulated for administration of about 1350 mg of the USP1 inhibitor or an amount of the salt, solvate, hydrate, or co-crystal thereof equivalent to about 1350 mg of the USP1 inhibitor. In some aspects, a medicament is formulated for administration of about 1400 mg of the USP1 inhibitor or an amount of the salt, solvate, hydrate, or co-crystal thereof equivalent to about 1400 mg of the USP1 inhibitor. In some aspects, a medicament is formulated for administration of about 1450 mg of the USP1 inhibitor or an amount of the salt, solvate, hydrate, or co-crystal thereof equivalent to about 1450 mg of the USP1 inhibitor. In some aspects, a medicament is formulated for administration of about 1500 mg of the USP1 inhibitor or an amount of the salt, solvate, hydrate, or co-crystal thereof equivalent to about 1500 mg of the USP1 inhibitor. In some aspects, a medicament is formulated for administration of about 1550 mg of the USP1 inhibitor or an amount of the salt, solvate, hydrate, or co-crystal thereof equivalent to about 1550 mg of the USP1 inhibitor. In some aspects, a medicament is formulated for administration of about 1600 mg of the USP1 inhibitor or an amount of the salt, solvate, hydrate, or co-crystal thereof equivalent to about 1600 mg of the USP1 inhibitor. In some aspects, a medicament is formulated for administration of about 1650 mg of the USP1 inhibitor or an amount of the salt, solvate, hydrate, or co-crystal thereof equivalent to about 1650 mg of the USP1 inhibitor. In some aspects, a medicament is formulated for administration of about 1700 mg of the USP1 inhibitor or an amount of the salt, solvate, hydrate, or co-crystal thereof equivalent to about 1700 mg of the USP1 inhibitor. In some aspects, a medicament is formulated for administration of about 1750 mg of the USP1 inhibitor or an amount of the salt, solvate, hydrate, or co-crystal thereof equivalent to about 1750 mg of the USP1 inhibitor. In some aspects, a medicament is formulated for administration of about 1800 mg of the USP1 inhibitor or an amount of the salt, solvate, hydrate, or co-crystal thereof equivalent to about 1800 mg of the USP1 inhibitor. In some aspects, a medicament is formulated for administration of about 1850 mg of the USP1 inhibitor or an amount of the salt, solvate, hydrate, or co-crystal thereof equivalent to about 1850 mg of the USP1 inhibitor. In some aspects, a medicament is formulated for administration of about 1900 mg of the USP1 inhibitor or an amount of the salt, solvate, hydrate, or co-crystal thereof equivalent to about 1900 mg of the USP1 inhibitor. In some aspects, a medicament is formulated for administration of about 1950 mg of the USP1 inhibitor or an amount of the salt, solvate, hydrate, or co-crystal thereof equivalent to about 1950 mg of the USP1 inhibitor. In some aspects, a medicament is formulated for administration of about 2000 mg of the USP1 inhibitor or an amount of the salt, solvate, hydrate, or co-crystal thereof equivalent to about 2000 mg of the USP1 inhibitor. In some aspects, a medicament is formulated for administration of about 2100 mg of the USP1 inhibitor or an amount of the salt, solvate, hydrate, or co-crystal thereof equivalent to about 2100 mg of the USP1 inhibitor. In some aspects, a medicament is formulated for administration of about 2200 mg of the USP1 inhibitor or an amount of the salt, solvate, hydrate, or co-crystal thereof equivalent to about 2200 mg of the USP1 inhibitor. In some aspects, a medicament is formulated for administration of about 2300 mg of the USP1 inhibitor or an amount of the salt, solvate, hydrate, or co-crystal thereof equivalent to about 2300 mg of the USP1 inhibitor. In some aspects, a medicament is formulated for administration of about 2400 mg of the USP1 inhibitor or an amount of the salt, solvate, hydrate, or co-crystal thereof equivalent to about 2400 mg of the USP1 inhibitor. In some aspects, a medicament is formulated for administration of about 2500 mg of the USP1 inhibitor or an amount of the salt, solvate, hydrate, or co-crystal thereof equivalent to about 2500 mg of the USP1 inhibitor. In some aspects, a medicament is formulated for administration of about 50 mg to about 2500 mg of the USP1 inhibitor or an amount of the salt, solvate, hydrate, or co-crystal thereof equivalent to about 50 mg to about 2500 mg of the USP1 inhibitor. In some aspects, a medicament is formulated for administration of about 50 mg to about 2000 mg of the USP1 inhibitor or an amount of the salt, solvate, hydrate, or co-crystal thereof equivalent to about 50 mg to about 200 mg of the USP1 inhibitor. In some aspects, a medicament is formulated for administration of about 50 mg to about 1500 mg of the USP1 inhibitor or an amount of the salt, solvate, hydrate, or co-crystal thereof equivalent to about 50 mg to about 1500 mg of the USP1 inhibitor. In some aspects, a medicament is formulated for administration of about 50 mg to about 1000 mg of the USP1 inhibitor or an amount of the salt, solvate, hydrate, or co-crystal thereof equivalent to about 50 mg to about 1000 mg of the USP1 inhibitor. In some aspects, a medicament is formulated for administration of about 100 mg to about 1000 mg of the USP1 inhibitor or an amount of the salt, solvate, hydrate, or co-crystal thereof equivalent to about 100 mg to about 1000 mg of the USP1 inhibitor. In some aspects, a medicament is formulated for administration of about 200 mg to about 800 mg of the USP1 inhibitor or an amount of the salt, solvate, hydrate, or co-crystal thereof equivalent to about 200 mg to about 800 mg of the USP1 inhibitor. In some aspects in the paragraph above, the USP1 inhibitor is Compound I. In some aspects described in the paragraph above, the USP1 inhibitor is administered as a divided dose over the course of a day. For example, in some aspects, a the USP1 inhibitor is administered over a course of two administrations in a day (e.g., if the dose is 1000 mg or equivalents thereof, the USP1 inhibitor is administered in a first dose of 500 mg and a second dose of 500 mg on the same day, wherein the first dose and the second dose are not simultaneous). The amount of the first dose and the second dose may be the same or different.

In some aspects, such a medicament is for administration about once per day. In some aspects, such a medicament is for administration at least twice (e.g., one two consecutive days). In some aspects, such a medicament is for administration at least three times (e.g., on three consecutive days). In some aspects, such a medicament is for administration at least four times (e.g., on four consecutive days). In some aspects, such a medicament is for administration at least five times (e.g., on five consecutive days). In some aspects, such a medicament is for administration at least five times (e.g., on five consecutive days). In some aspects, such a medicament is for administration at least 10 times (e.g., on about 10 consecutive days). In some aspects, such a medicament is for administration at least 15 times (e.g., on about 15 consecutive days). In some aspects, such a medicament is for administration at least 20 times (e.g., on about 20 consecutive days). In some aspects, such a medicament is for administration at least 25 times (e.g., on about 25 consecutive days). In some aspects, such a medicament is for administration about 28 times (e.g., on about 28 consecutive days). In some aspects, such a medicament is for administration for a period of at least 3 days, or at least 4 days, or at least 5 days, or at least 10 days, or at least 15 days, or at least 20 days, or at least 25 days, or at least 1 month, or at least 2 months, or at least 3 months, or at least 4 months, or at least 5 months, or at least 6 months, or at least 7 months, or at least 8 months, or at least 9 months, or at least 10 months, or at least 11 months, or at least 1 year, or at least 18 months, or at least 2 years, or more.

In some aspects, such a medicament is for administration about once every 21 days (about once every three weeks. In some aspects, such a medicament is for administration at least twice (e.g., about 21 days apart). In some aspects, such a medicament is for administration at least three times (e.g., about 21 days apart). In some aspects, such a medicament is for administration at least four times (e.g., about 21 days apart). In some aspects, such a medicament is for administration at least five times (e.g., about 21 days apart).

In some aspects, the cancer to be treated with a USP1 inhibitor (e.g., Compound I or a pharmaceutically acceptable salt, solvate, hydrate, or co-crystal thereof) is a solid tumor. In some aspects, the cancer to be treated with a USP1 inhibitor is a cancer that comprises cancer cells with a mutation in a gene encoding p53. In some aspects, the cancer to be treated with a USP1 inhibitor is a cancer that comprises cancer cells with a loss of function mutation in a gene encoding p53. In some aspects, the cancer to be treated with a USP1 inhibitor is a cancer that comprises cancer cells with a mutation in a gene encoding BRCA1 (a somatic and/or germline mutation). In some aspects, the cancer to be treated with a USP1 inhibitor is a cancer that comprises cancer cells with a mutation in a gene encoding BRCA2 (a somatic and/or germline mutation). In some aspects, the cancer to be treated with a USP1 inhibitor is a cancer that comprises cancer cells with a loss of function mutation in a gene encoding ATM.

In some aspects, the cancer to be treated with a USP1 inhibitor is selected from lung cancer (including non-small cell lung cancer (NSCLC)), colon cancer, bladder cancer, osteosarcoma, ovarian cancer, skin cancer, and breast cancer. In some aspects, the cancer to be treated with a USP1 inhibitor is selected from lung cancer non-small cell lung cancer (NSCLC), osteosarcoma, ovarian cancer, and breast cancer. In some aspects, the cancer is ovarian cancer or breast cancer. In some aspects, the cancer is ovarian cancer. In some aspects, the ovarian cancer is serous ovarian cancer. In some aspects, the ovarian cancer has previously been treated with platinum-based chemotherapy. In some aspects, the ovarian cancer is platinum-resistant. In some aspects, the ovarian cancer is platinum-sensitive. In some aspects, the cancer is breast cancer. In some aspects, the cancer is a triple negative breast cancer. In some aspects, the breast cancer is human epidermal growth factor receptor 2 (HER2) negative. In some aspects, the breast cancer is germline breast cancer susceptibility gene mutation (gBRCAm) breast cancer. As used herein gBRCAm breast cancer refers to a breast cancer with a documented mutation in BRCA1 or BRCA2 that is predicted to be deleterious or suspected deleterious (known or predicted to be detrimental/lead to loss of function). In some aspects, the breast cancer is metastatic breast cancer.

In some aspects, the cancer to be treated with a USP1 inhibitor is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

In some aspects, the cancer is a recurrent cancer. In some aspects, cancer to be treated with a USP1 inhibitor has previously been treated with a PARP inhibitor (PARPi). In some aspects, the cancer to be treated with a USP1 inhibitor has previously been treated with at least one systemic chemotherapy for metastatic disease.

In some aspects, the cancer to be treated with a USP1 inhibitor comprises cancer cells with elevated levels of RAD18. In some aspects, the elevated levels of RAD18 are elevated RAD18 protein levels. In some aspects, the elevated levels of RAD18 are elevated RAD18 mRNA levels. In some aspects, elevated levels of RAD18 (e.g., RAD18 protein and/or RAD18 mRNA) have been detected (e.g., in a cancer sample obtained from the subject) prior to the administration. That is, in some aspects, a subject's cancer has been tested for RAD18 protein or mRNA prior to beginning treatment with a USP1 inhibitor.

In some aspects, the cancer to be treated with a USP1 inhibitor is a cancer that comprises cancer cells with elevated levels of RAD51. The elevated levels of RAD51 can be elevated RAD51 protein levels, elevated RAD51 protein foci levels, and/or elevated levels RAD51 mRNA levels. In some aspects, a cancer that comprises cancer cells with elevated levels of RAD51 refers to a cancer wherein at least 10% of cells that are in the S/G2 phase of the cell cycle (e.g., geminin-positive cells) in a sample obtained from the cancer are RAD51-positive (e.g., contain 5 or more RAD51 nuclear foci).

A cancer with elevated levels of RAD51 can be a homologous-recombination deficient cancer. A cancer with elevated levels of RAD51 can be a BRCA1 mutant cancer, a BRCA2 mutant cancer, or a BRCA1 and BRCA2 mutant cancer. A cancer with elevated levels of RAD51 can be cancer with deleterious or suspected deleterious mutations in BRCA1 and BRCA2 genes and/or a positive Genomic Instability Score, e.g., as determined using myChoice© CDx (Myriad©).

Cancers comprising cells with elevated levels of RAD51 can be referred to herein as “RAD51 high cancers.”

RAD51 protein levels can be detected using, for example, immunofluorescence, western blots, fluorescence-activated cell sorting (FACS), and/or immunohistochemistry. RAD51 mRNA levels can be detected, for example, using quantitative reverse transcriptase (RT)-polymerase chain reaction (PCR). Elevated levels of RAD51 protein and/or mRNA indicate that a cancer is sensitive to USP1 inhibitors.

Methods of detecting RAD51 and RAD51 protein foci are provided, for example, in Castroviejo-Bermejo, Marta, et al., EMBO Molecular Medicine 10(12):e9172 (2018), which is herein incorporated by reference in its entirety. RAD51 can be detected, for example, using immunofluorescence. RAD51 foci, e.g., of 0.42-1.15 μm diameter can be quantified on formalin-fixed paraffin embedded (FFPE) tumor samples, by scoring the percentage of cells in the S/G2-cell cycle phase (e.g., geminin-positive cells) with 5 or more RAD51 nuclear foci. In some aspects, cancers comprising cells with elevated levels of RAD51 are cancers wherein at least 10% of cells that are in the S/G2 phase of the cell cycle (e.g., geminin-positive cells) are RAD51-positive.

In some aspects, a USP1 inhibitor is used to treat a cancer, wherein the cancer is a homologous-recombination deficient cancer. In some aspects, a USP1 inhibitor is used to treat a cancer, wherein the cancer comprises cancer cells with a mutation in a gene encoding p53. In some aspects, a USP1 inhibitor is used to treat a cancer, wherein the cancer comprises cancer cells with a loss of function mutation in a gene encoding p53. In some aspects, a USP1 inhibitor is used to treat a cancer that does not have a defect in the homologous recombination pathway.

In some aspects, a USP1 inhibitor is used to treat a cancer, wherein the cancer is a BRCA1 (somatic or germline) mutant cancer. In some aspects, a USP1 inhibitor is used to treat a cancer, wherein the cancer is a BRCA2 (somatic or germline) mutant cancer. In some aspects, a USP1 inhibitor is used to treat a cancer, wherein the cancer is a BRCA1 (somatic or germline) mutant cancer and a BRCA2 (somatic or germline) mutant cancer. In some aspects, the cancer is not a BRCA1 mutant cancer or a BRCA2 mutant cancer. In some aspects, the cancer is a BRCA1 deficient cancer. In some aspects, the cancer is a BRCA2 deficient cancer. In some aspects, the cancer is a BRCA1 deficient cancer and a BRCA2 deficient cancer.

In some aspects, a USP1 inhibitor is used to treat a cancer, wherein the cancer is an ATM mutant cancer. In some aspects, the cancer is not an ATM mutant cancer. In some aspects, the cancer is an ATM deficient cancer.

In some aspects, a USP1 inhibitor is used to treat a cancer, wherein the cancer is a PARP inhibitor resistant or refractory cancer. In some aspects, a USP1 inhibitor is used to treat a cancer, wherein the cancer is a PARP inhibitor resistant or refractory BRCA1-deficient cancer.

In some aspects, the cancer is a BRCA1 and/or BRCA2 mutant cancer, wherein the cancer comprises cells with elevated levels of RAD18, e.g., wherein the elevated levels of RAD1S are at least as high as the RAD18 protein and/or mRNA levels in ES2 cells or wherein the elevated levels of RAD18 are higher than the RAD18 protein and/or mRNA levels in HEP3B217 cells. In some aspects, a triple negative breast cancer is a BRCA1 and/or BRCA2 mutant cancer.

In some aspects, the cancer is a solid cancer. In some aspects, the cancer is a DNA damage repair pathway deficient cancer. In some aspects, the cancer is a homologous-recombination deficient cancer. In some aspects, the cancer comprises cancer cells with a mutation in a gene encoding p53. In some aspects, the cancer comprises cancer cells with a loss of function mutation in a gene encoding p53. In some aspects, the cancer is selected from the group consisting of non-small cell lung cancer (NSCLC), ovarian cancer, and breast cancer (including triple negative breast cancer). In some aspects, the cancer is ovarian cancer or breast cancer (including triple negative breast cancer). In some aspects, the cancer is ovarian cancer (including serous ovarian cancer). In some aspects, the ovarian cancer has previously been treated with platinum-based chemotherapy. In some aspects, the ovarian cancer is platinum-resistant. In some aspects, the cancer is breast cancer (including triple negative breast cancer). In some aspects, the breast cancer is human epidermal growth factor receptor 2 (HER2) negative. In some aspects, the breast cancer is germline breast cancer susceptibility gene mutation (gBRCAm) breast cancer. In some aspects, the breast cancer is metastatic breast cancer.

### III. USP1 Inhibitors

USP1 inhibitors have been disclosed, for example, in WO2020/132269 and PCT/US2021/057072, each of which is herein incorporated by reference in its entirety. In some aspects provided herein, a USP1 inhibitor is 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine or “Compound I”

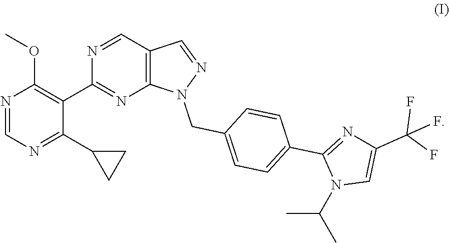 [SMILES: COc1ncnc(C2CC2)c1-c1ncc2cnn(Cc3ccc(-c4nc(C(F)(F)F)cn4C(C)C)cc3)c2n1]

or a pharmaceutically acceptable salt, solvate, hydrate, or co-crystal thereof.

In some aspects, a USP1 inhibitor is a solid state form of a compound of Formula (I) or a pharmaceutically acceptable salt, solvate, hydrate, or co-crystal thereof. In some aspects, the solid state form is an amorphous form of a compound of Formula (I). In some aspects, the amorphous form is a hydrate, anhydrate, or solvate thereof. In some aspects, the amorphous form is substantially free of other polymorphic forms.

In some aspects, a USP1 inhibitor comprises a mixture comprising a majority of the amorphous form as compared to other solid state forms of a compound of Formula (I). In some aspects, the amorphous form has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In some aspects, the solid state form is a crystalline form of a compound of Formula (I). In some aspects, the crystalline form is a hydrate, anhydrate, or solvate thereof. In some aspects, the solvate is a dichloromethane solvate.

In some aspects, a USP1 inhibitor is a solid state form of a compound of Formula (I) in crystalline Form A, wherein Form A is characterized by an XRPD pattern having peaks at 14.3±0.2, 21.5±0.2, and 21.8±0.2 degrees two theta.

The sections below discuss solid state forms of a compound of Formula (I) that have been identified and selected properties of those solid state forms.

### A. Crystalline Form A

In some aspects, a USP1 inhibitor for the uses and methods provided herein is a crystalline Form A of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula MI.

In some aspects, crystalline Form A is a 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine hydrate.

In some aspects, the melting point of crystalline Form A is about 165° C.

In some aspects, crystalline Form A is characterized by an XRPD pattern having peaks at 14.3±0.2, 21.5±0.2, and 21.8±0.2 degrees two theta when measured by Cu Kα radiation. In some aspects, crystalline Form A is characterized by an XRPD pattern having peaks at 7.1±0.2, 14.3±0.2, 21.5±0.2, and 21.8±0.2 degrees two theta when measured by Cu Kα radiation. In some aspects, crystalline Form A is characterized by an XRPD pattern having peaks at 7.1±0.2, 14.3±0.2, 19.1±0.2, 21.5±0.2, and 21.8±0.2 degrees two theta when measured by Cu Kα radiation. In some aspects, crystalline Form A is characterized by an XRPD pattern having peaks at 7.1 0.2, 14.3 0.2, 15.2 0.2, 19.1±0.2, 21.5±0.2, and 21.8±0.2 degrees two theta when measured by Cu Kα radiation.

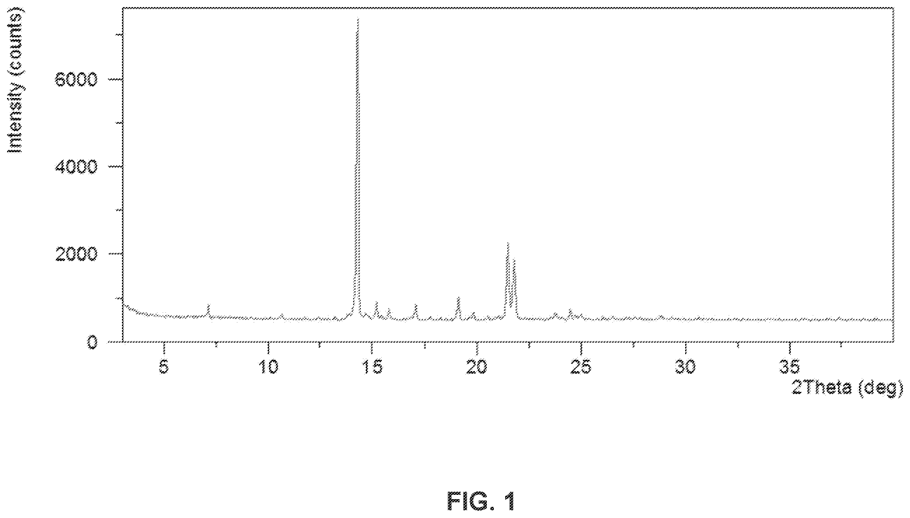

In some aspects, crystalline Form A is characterized by an XRPD pattern substantially as shown in FIG. 1.

In some aspects, crystalline Form A is characterized by three or more, four or more, five or more, or six or more XRPD peaks listed in Table 1 (a and b) below.

TABLE 1a
Selected XRPD Peaks for Crystalline Form A

| Pos. [°2Th.] | d-spacing [Å] | Rel. Int. [%] |
|---|---|---|
| 7.14 | 12.39 | 4.09 |
| 14.29 | 6.20 | 100.00 |
| 15.20 | 5.83 | 5.85 |
| 17.08 | 5.19 | 5.08 |
| 19.13 | 4.64 | 7.52 |
| 21.49 | 4.13 | 26.71 |
| 21.80 | 4.08 | 20.57 |

TABLE 1b
XRPD Peaks for Crystalline Form A

| Pos. [°2Th.] | d-spacing [Å] | Rel. Int. [%] |
|---|---|---|
| 7.14 | 12.39 | 4.09 |
| 10.64 | 8.32 | 1.01 |
| 14.29 | 6.20 | 100.00 |
| 15.20 | 5.83 | 5.85 |
| 15.80 | 5.61 | 3.61 |
| 17.08 | 5.19 | 5.08 |
| 17.74 | 5.00 | 0.83 |
| 19.13 | 4.64 | 7.52 |
| 19.84 | 4.48 | 2.39 |
| 21.49 | 4.13 | 26.71 |
| 21.80 | 4.08 | 20.57 |
| 23.78 | 3.74 | 1.78 |
| 24.47 | 3.64 | 3.29 |
| 25.00 | 3.56 | 1.42 |
| 28.82 | 3.10 | 1.11 |

In some aspects, crystalline Form A is characterized by an endothermic peak at from about 162° C. to about 168° C., or from about 163° C. to about 167° C., or from about 164° C. to about 166° C., as determined by DSC. In some aspects, crystalline Form A is characterized by an endothermic peak at about 165° C., as determined by DSC.

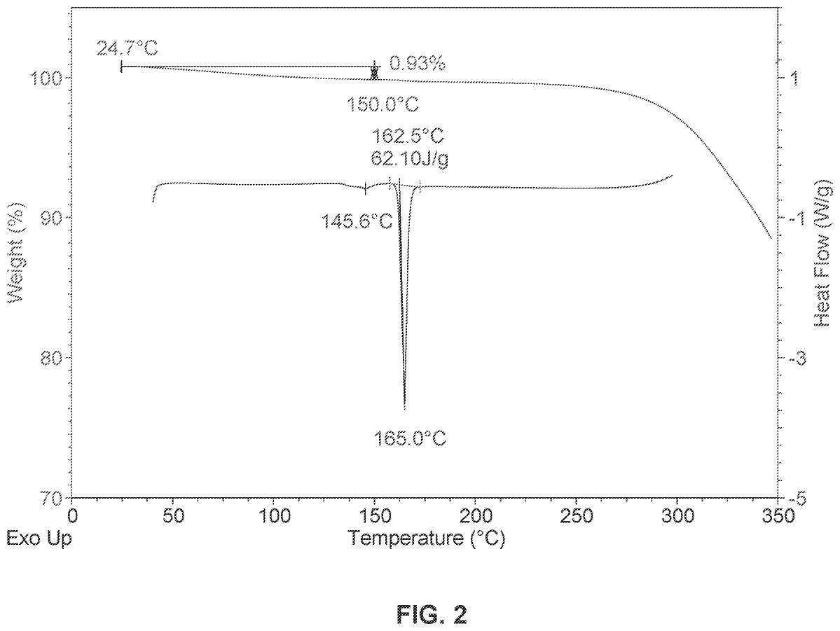

In some aspects, crystalline Form A is characterized by a DSC profile substantially as shown in FIG. 2.

In some aspects, crystalline Form A is characterized by from an about 0.88 wt % to an about 0.98 wt % loss between room temperature and about 150° C. In some aspects, crystalline Form A is characterized by from an about 0.90 wt % to an about 0.96 wt % loss between room temperature and about 150° C. In some aspects, crystalline Form A is characterized by from an about 0.93 wt % loss between room temperature and about 150° C.

In some aspects, crystalline Form A is characterized by a TGA profile substantially as shown in FIG. 2.

In some aspects, crystalline Form A is characterized by at least two of the following: a) an XRPD pattern as shown in FIG. 1; b) a DSC profile as shown in FIG. 2; or c) a TGA profile as shown in FIG. 2.

In some aspects, crystalline Form A has a unit cell that indexes as monoclinic.

In some aspects, crystalline Form A has a unit cell with an a value of about 12.054 Å, a b value of about 8.775 Å, and a c value of about 24.837 Å. In some aspects, Form A has a unit cell with a volume of about 2603.68 Å³.

In some aspects, crystalline Form A is substantially free of other polymorphic forms. In some aspects, crystalline Form A has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%. In some aspects, crystalline Form A has a polymorphic purity of at least 80%.

In another aspect, the present disclosure relates to a mixture comprising crystalline Form A and a second solid state form of a compound of Formula (I). In some aspects, the second solid state form of a compound of Formula (I) is crystalline Form 2.

In some aspects, the present disclosure relates to a mixture comprising a majority of crystalline Form A as compared to other solid state forms of a compound of Formula (I).

### B. Crystalline Form 2

In some aspects, a USP1 inhibitor for the uses and methods provided herein is a solid state form that is a pharmaceutically acceptable salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I). In some aspects, the pharmaceutically acceptable salt is formed between 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I) and a pharmaceutically acceptable acid. In some aspects, the pharmaceutically acceptable acid is gentisic acid.

In some aspects, a USP1 inhibitor is a gentisate salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I) in an amorphous form. In some aspects, the amorphous form is substantially free of other polymorphic forms. In some aspects, the amorphous form has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In some aspects, a USP1 inhibitor is a gentisate salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I) in a co-crystalline form.

In some aspects, a USP1 inhibitor is a solid state form of a pharmaceutically acceptable gentisate co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I) and a second pharmaceutically acceptable compound. The pharmaceutically acceptable co-crystal is formed between 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I) and gentisic acid.

In some aspects, the gentisic acid co-crystal is crystalline Form 2 characterized by an XRPD pattern having peaks at 16.6±0.2, 18.7±0.2, and 22.5±0.2 degrees two theta.

In some aspects, crystalline Form 2 is an anhydrate.

In some aspects, the melting point of crystalline Form 2 is from about 184° C. to about 190° C. In some aspects, the melting point of crystalline Form 2 is from about 186° C. to about 188° C. In some aspects, the melting point of crystalline Form 2 is about 187° C.

In some aspects, crystalline Form 2 is characterized by an XRPD pattern having peaks at 16.6±0.2, 18.7±0.2, and 22.5±0.2 degrees two theta when measured by Cu Kα radiation. In another aspect, crystalline Form 2 is characterized by an XRPD pattern having peaks at 16.6±0.2, 18.7±0.2, 22.3±0.2, and 22.5±0.2 degrees two theta when measured by Cu Kα radiation. In some aspects, crystalline Form 2 is characterized by an XRPD pattern having peaks at 16.6±0.2, 18.7±0.2, 22.3±0.2, 22.5±0.2, and 26.0±0.2 degrees two theta when measured by Cu Kα radiation. In some aspects, crystalline Form 2 is characterized by an XRPD pattern having peaks at 16.6±0.2, 18.7±0.2, 20.8±0.2, 22.3±0.2, 22.5±0.2, and 26.0±0.2 degrees two theta when measured by Cu Kα radiation.

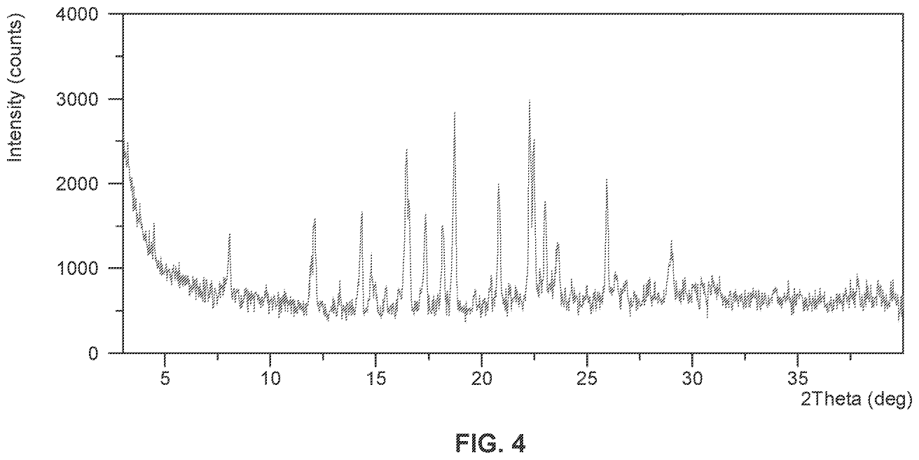

In some aspects, crystalline Form 2 is characterized by an XRPD pattern substantially as shown in FIG. 4. In some aspects, crystalline Form 2 is characterized by three or more, four or more, five or more, or six or more XRPD peaks listed in Table 2 (a and b) below:

TABLE 2a
Select XRPD Peaks for Crystalline Form 2.

| Pos. [°2Th.] | d-spacing [Å] | Rel. Int. [%] |
|---|---|---|
| 8.06 | 10.97 | 21.93 |
| 12.12 | 7.30 | 23.48 |
| 13.30 | 6.66 | 9.94 |
| 14.31 | 6.19 | 37.35 |
| 14.95 | 5.93 | 21.37 |
| 15.47 | 5.73 | 12.00 |
| 16.42 | 5.40 | 38.25 |
| 16.58 | 5.35 | 59.48 |
| 17.35 | 5.11 | 18.11 |
| 18.18 | 4.88 | 30.28 |
| 18.72 | 4.74 | 61.10 |
| 19.68 | 4.51 | 5.01 |
| 20.40 | 4.35 | 6.62 |
| 20.80 | 4.27 | 37.19 |
| 22.29 | 3.99 | 95.77 |
| 22.49 | 3.95 | 100.00 |
| 23.00 | 3.87 | 21.16 |
| 23.65 | 3.76 | 19.85 |
| 25.96 | 3.43 | 38.67 |
| 26.40 | 3.38 | 7.96 |
| 26.88 | 3.32 | 8.15 |
| 28.95 | 3.08 | 8.97 |
| 30.06 | 2.97 | 6.51 |

TABLE 2b
XRPD Peaks for Crystalline Form 2

| Pos. [°2Th.] | d-spacing [Å] | Rel. Int. |
|---|---|---|
| 8.06 | 10.97 | 21.93 |
| 12.12 | 7.30 | 23.48 |
| 13.30 | 6.66 | 9.94 |
| 14.31 | 6.19 | 37.35 |
| 14.95 | 5.93 | 21.37 |
| 15.47 | 5.73 | 12.00 |
| 16.42 | 5.40 | 38.25 |
| 16.58 | 5.35 | 59.48 |
| 17.35 | 5.11 | 18.11 |
| 18.18 | 4.88 | 30.28 |
| 18.72 | 4.74 | 61.10 |
| 19.68 | 4.51 | 5.01 |
| 20.40 | 4.35 | 6.62 |
| 20.80 | 4.27 | 37.19 |
| 22.29 | 3.99 | 95.77 |
| 22.49 | 3.95 | 100.00 |
| 23.00 | 3.87 | 21.16 |
| 23.65 | 3.76 | 19.85 |
| 25.96 | 3.43 | 38.67 |
| 26.40 | 3.38 | 7.96 |
| 26.88 | 3.32 | 8.15 |
| 28.95 | 3.08 | 8.97 |
| 30.06 | 2.97 | 6.51 |
| 31.12 | 2.87 | 2.70 |
| 34.18 | 2.62 | 1.65 |

In some aspects, crystalline Form 2 is characterized by an endothermic peak at from about 181° C. to about 191° C., or from about 183° C. to about 189° C., or from about 185° C. to about 187° C., as determined by DSC. In some aspects, crystalline Form 2 is characterized by an endothermic peak at about 186.0° C., as determined by DSC.

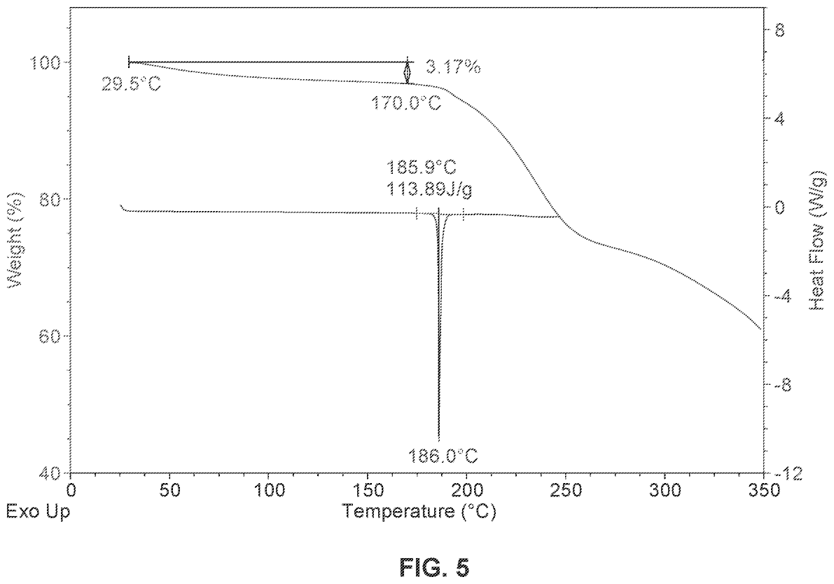

In some aspects, crystalline Form 2 is characterized by a DSC profile substantially as shown in FIG. 5.

In some aspects, crystalline Form 2 is characterized by from an about 2.5 wt % to an about 3.5 wt % loss between room temperature and about 170° C. In some aspects, crystalline Form 2 is characterized by from an about 3.0 wt % to an about 3.4 wt % loss between room temperature and about 170° C. In some aspects, crystalline Form 2 is characterized by an about 3.17 wt % loss between room temperature and about 170° C.

In some aspects, crystalline Form 2 is characterized by a TGA profile substantially as shown in FIG. 5.

In some aspects, crystalline Form 2 is characterized by at least two of the following: a) an XRPD pattern as shown in FIG. 4; b) a DSC profile as shown in FIG. 5; or c) a TGA profile as shown in FIG. 5.

In some aspects, crystalline Form 2 has a unit cell that indexes as monoclinic.

In some aspects, crystalline Form 2 has a unit cell with an a value of about 11.113 Å, a b value of about 12.356 Å, and a c value of about 24.048 Å. In some aspects, Form 2 has a unit cell with a volume of about 3223.93 Å³.

The unit cell parameters for crystalline Form 2 shown in Table 3.

TABLE 3
Unit Cell Parameters for Crystalline Form 2.

| Crystal System | Monoclinic |
|---|---|
| a [Å] | 11.113 |
| b [Å] | 12.356 |
| c [Å] | 24.048 |
| α [deg] | 90 |
| β [deg] | 102.48 |
| γ [deg] | 90 |
| Volume [Å³] | 3223.93 |
| Z CalMolated density | 4, 1.419 |
| [g/cm³] | |
| Crystal Size [mm³] | 0.31 × 0.15 × 0.11 |
| Space Group(s) | P2_{1/c} |

In some aspects, crystalline Form 2 is substantially free of other polymorphic forms. In some aspects, crystalline Form 2 has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In some aspects, the a USP1 inhibitor comprises a mixture comprising crystalline Form 2 and a second solid state form of a compound of Formula (I). In some aspects, the second solid state form of a compound of Formula (I) is crystalline Form A. In some aspects, a USP1 inhibitor comprises a mixture comprising a majority of crystalline Form 2 as compared to other solid state forms of a compound of Formula (I).

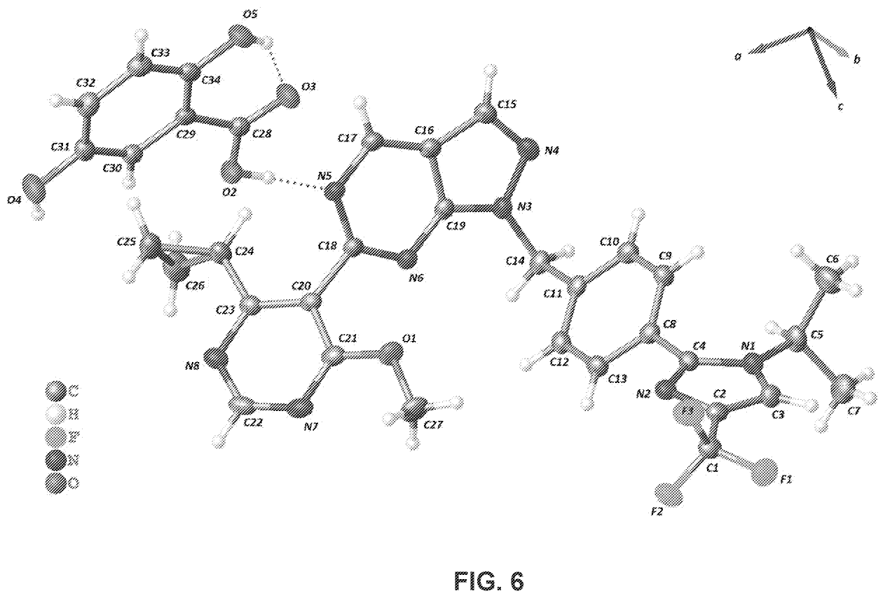

Crystalline Form 2 exhibits chemical and physical properties that are unexpected and bioavailablity properties that are advantageous compared to the free base forms. In particular, as shown in FIG. 6, 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I) and gentisic acid exhibit significant hydrogen bonding interactions despite being an interaction between a weak base and a weak acid, respectively.

Additionally, as shown in Example 3, crystalline Form 2 surprisingly exhibits increased mouse oral exposure levels as compared to other solid state forms of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I). For example, as shown in Example 1, in some aspects, crystalline Form 2 (gentisic acid co-crystal) exhibits higher exposure levels at about 300 mg/kg than crystalline Form A (freebase).

### III. Pharmaceutical Compositions and Kits

As provided herein, a USP1 inhibitor (e.g., Compound I or a pharmaceutically acceptable salt, solvate, hydrate, or co-crystal thereof) can be in any pharmaceutical composition including all compositions where such an inhibitor is combined with one or more pharmaceutically acceptable carriers. In some aspects, the USP1 inhibitor is present in a composition in an amount that is effective to achieve its intended therapeutic purpose.

In some aspects, a pharmaceutical composition comprises about 75 mg to about 1000 mg of a USP1 inhibitor (e.g., Compound I) or a pharmaceutically acceptable salt, solvate, hydrate, or co-crystal thereof in an amount equivalent to about 75 mg to about 1000 mg of the USP1 inhibitor. In some aspects, a pharmaceutical composition comprises about 75 mg of a USP1 inhibitor (e.g., Compound I) or a pharmaceutically acceptable salt, solvate, hydrate, or co-crystal thereof in an amount equivalent to about 75 mg of the USP1 inhibitor. In some aspects, a pharmaceutical composition comprises about 100 mg of a USP1 inhibitor (e.g., Compound I) or a pharmaceutically acceptable salt, solvate, hydrate, or co-crystal thereof in an amount equivalent to about 100 mg of the USP1 inhibitor. In some aspects, a pharmaceutical composition comprises about 150 mg of a USP1 inhibitor (e.g., Compound I) or a pharmaceutically acceptable salt, solvate, hydrate, or co-crystal thereof in an amount equivalent to about 150 mg of the USP1 inhibitor. In some aspects, a pharmaceutical composition comprises about 200 mg of a USP1 inhibitor (e.g., Compound I) or a pharmaceutically acceptable salt, solvate, hydrate, or co-crystal thereof in an amount equivalent to about 200 mg of the USP1 inhibitor. In some aspects, a pharmaceutical composition comprises about 250 mg of a USP1 inhibitor (e.g., Compound 1) or a pharmaceutically acceptable salt, solvate, hydrate, or co-crystal thereof in an amount equivalent to about 250 mg of the USP1 inhibitor. In some aspects, a pharmaceutical composition comprises about 300 mg of a USP1 inhibitor (e.g., Compound I) or a pharmaceutically acceptable salt, solvate, hydrate, or co-crystal thereof in an amount equivalent to about 300 mg of the USP1 inhibitor. In some aspects, a pharmaceutical composition comprises about 350 mg of a USP1 inhibitor (e.g., Compound I) or a pharmaceutically acceptable salt, solvate, hydrate, or co-crystal thereof in an amount equivalent to about 350 mg of the USP1 inhibitor. In some aspects, a pharmaceutical composition comprises about 400 mg of a USP1 inhibitor (e.g., Compound I) or a pharmaceutically acceptable salt, solvate, hydrate, or co-crystal thereof in an amount equivalent to about 400 mg of the USP1 inhibitor. In some aspects, a pharmaceutical composition comprises about 450 mg of a USP1 inhibitor (e.g., Compound I) or a pharmaceutically acceptable salt, solvate, hydrate, or co-crystal thereof in an amount equivalent to about 450 mg of the USP1 inhibitor. In some aspects, a pharmaceutical composition comprises about 500 mg of a USP1 inhibitor (e.g., Compound I) or a pharmaceutically acceptable salt, solvate, hydrate, or co-crystal thereof in an amount equivalent to about 500 mg of the USP1 inhibitor. In some aspects, a pharmaceutical composition comprises about 550 mg of a USP1 inhibitor (e.g., Compound I) or a pharmaceutically acceptable salt, solvate, hydrate, or co-crystal thereof in an amount equivalent to about 550 mg of the USP1 inhibitor. In some aspects, a pharmaceutical composition comprises about 600 mg of a USP1 inhibitor (e.g., Compound I) or a pharmaceutically acceptable salt, solvate, hydrate, or co-crystal thereof in an amount equivalent to about 600 mg of the USP1 inhibitor. In some aspects, a pharmaceutical composition comprises about 650 mg of a USP1 inhibitor (e.g., Compound I) or a pharmaceutically acceptable salt, solvate, hydrate, or co-crystal thereof in an amount equivalent to about 650 mg of the USP1 inhibitor. In some aspects, a pharmaceutical composition comprises about 700 mg of a USP1 inhibitor (e.g., Compound I) or a pharmaceutically acceptable salt, solvate, hydrate, or co-crystal thereof in an amount equivalent to about 700 mg of the USP1 inhibitor. In some aspects, a pharmaceutical composition comprises about 750 mg of a USP1 inhibitor (e.g., Compound I) or a pharmaceutically acceptable salt, solvate, hydrate, or co-crystal thereof in an amount equivalent to about 750 mg of the USP1 inhibitor. In some aspects, a pharmaceutical composition comprises about 800 mg of a USP1 inhibitor (e.g., Compound I) or a pharmaceutically acceptable salt, solvate, hydrate, or co-crystal thereof in an amount equivalent to about 800 mg of the USP1 inhibitor. In some aspects, a pharmaceutical composition comprises about 850 mug of a USP1 inhibitor (e.g., Compound I) or a pharmaceutically acceptable salt, solvate, hydrate, or co-crystal thereof in an amount equivalent to about 850 mg of the USP1 inhibitor. In some aspects, a pharmaceutical composition comprises about 900 mg of a USP1 inhibitor (e.g., Compound I) or a pharmaceutically acceptable salt, solvate, hydrate, or co-crystal thereof in an amount equivalent to about 900 mg of the USP1 inhibitor. In some aspects, a pharmaceutical composition comprises about 950 mg of a USP1 inhibitor (e.g., Compound I) or a pharmaceutically acceptable salt, solvate, hydrate, or co-crystal thereof in an amount equivalent to about 950 mg of the USP1 inhibitor. In some aspects, a pharmaceutical composition comprises about 1000 mg of a USP1 inhibitor (e.g., Compound I) or a pharmaceutically acceptable salt, solvate, hydrate, or co-crystal thereof in an amount equivalent to about 1000 mg of the USP1 inhibitor. In some aspects, a pharmaceutical composition comprises about 1050 mg of a USP1 inhibitor (e.g., Compound I) or a pharmaceutically acceptable salt, solvate, hydrate, or co-crystal thereof in an amount equivalent to about 1050 mg of the USP1 inhibitor. In some aspects, a pharmaceutical composition comprises about 1100 mg of a USP1 inhibitor (e.g., Compound I) or a pharmaceutically acceptable salt, solvate, hydrate, or co-crystal thereof in an amount equivalent to about 1100 mg of the USP1 inhibitor. In some aspects, a pharmaceutical composition comprises about 1150 mg of a USP1 inhibitor (e.g., Compound I) or a pharmaceutically acceptable salt, solvate, hydrate, or co-crystal thereof in an amount equivalent to about 1150 mg of the USP1 inhibitor. In some aspects, a pharmaceutical composition comprises about 1200 mg of a USP1 inhibitor (e.g., Compound I) or a pharmaceutically acceptable salt, solvate, hydrate, or co-crystal thereof in an amount equivalent to about 1200 mg of the USP1 inhibitor. In some aspects, a pharmaceutical composition comprises about 1250 mg of a USP1 inhibitor (e.g., Compound I) or a pharmaceutically acceptable salt, solvate, hydrate, or co-crystal thereof in an amount equivalent to about 1250 mg of the USP1 inhibitor. In some aspects, a pharmaceutical composition comprises about 1300 mg of a USP1 inhibitor (e.g., Compound I) or a pharmaceutically acceptable salt, solvate, hydrate, or co-crystal thereof in an amount equivalent to about 1300 mg of the USP1 inhibitor. In some aspects, a pharmaceutical composition comprises about 1350 mg of a USP1 inhibitor (e.g., Compound I) or a pharmaceutically acceptable salt, solvate, hydrate, or co-crystal thereof in an amount equivalent to about 1350 mg of the USP1 inhibitor. In some aspects, a pharmaceutical composition comprises about 1400 mg of a USP1 inhibitor (e.g., Compound I) or a pharmaceutically acceptable salt, solvate, hydrate, or co-crystal thereof in an amount equivalent to about 1400 mg of the USP1 inhibitor. In some aspects, a pharmaceutical composition comprises about 1450 mg of a USP1 inhibitor (e.g., Compound I) or a pharmaceutically acceptable salt, solvate, hydrate, or co-crystal thereof in an amount equivalent to about 1450 mg of the USP1 inhibitor. In some aspects, a pharmaceutical composition comprises about 1500 mg of a USP1 inhibitor (e.g., Compound I) or a pharmaceutically acceptable salt, solvate, hydrate, or co-crystal thereof in an amount equivalent to about 1500 mg of the USP1 inhibitor. In some aspects, a pharmaceutical composition comprises about 1550 mg of a USP1 inhibitor (e.g., Compound I) or a pharmaceutically acceptable salt, solvate, hydrate, or co-crystal thereof in an amount equivalent to about 1550 mg of the USP1 inhibitor. In some aspects, a pharmaceutical composition comprises about 1600 mg of a USP1 inhibitor (e.g., Compound I) or a pharmaceutically acceptable salt, solvate, hydrate, or co-crystal thereof in an amount equivalent to about 1600 mg of the USP1 inhibitor. In some aspects, a pharmaceutical composition comprises about 1650 mg of a USP1 inhibitor (e.g., Compound I) or a pharmaceutically acceptable salt, solvate, hydrate, or co-crystal thereof in an amount equivalent to about 1650 mg of the USP1 inhibitor. In some aspects, a pharmaceutical composition comprises about 1700 mg of a USP1 inhibitor (e.g., Compound I) or a pharmaceutically acceptable salt, solvate, hydrate, or co-crystal thereof in an amount equivalent to about 1700 mg of the USP1 inhibitor. In some aspects, a pharmaceutical composition comprises about 1750 mg of a USP1 inhibitor (e.g., Compound I) or a pharmaceutically acceptable salt, solvate, hydrate, or co-crystal thereof in an amount equivalent to about 1750 mg of the USP1 inhibitor. In some aspects, a pharmaceutical composition comprises about 1800 mg of a USP1 inhibitor (e.g., Compound I) or a pharmaceutically acceptable salt, solvate, hydrate, or co-crystal thereof in an amount equivalent to about 1800 mg of the USP1 inhibitor. In some aspects, a pharmaceutical composition comprises about 1850 mg of a USP1 inhibitor (e.g., Compound I) or a pharmaceutically acceptable salt, solvate, hydrate, or co-crystal thereof in an amount equivalent to about 1850 mg of the USP1 inhibitor. In some aspects, a pharmaceutical composition comprises about 1900 mg of a USP1 inhibitor (e.g., Compound I) or a pharmaceutically acceptable salt, solvate, hydrate, or co-crystal thereof in an amount equivalent to about 1900 mg of the USP1 inhibitor. In some aspects, a pharmaceutical composition comprises about 1950 mg of a USP1 inhibitor (e.g., Compound I) or a pharmaceutically acceptable salt, solvate, hydrate, or co-crystal thereof in an amount equivalent to about 1950 mg of the USP1 inhibitor. In some aspects, a pharmaceutical composition comprises about 2000 mg of a USP1 inhibitor (e.g., Compound I) or a pharmaceutically acceptable salt, solvate, hydrate, or co-crystal thereof in an amount equivalent to about 2000 mg of the USP1 inhibitor. In some aspects, a pharmaceutical composition comprises about 2100 mg of a USP1 inhibitor (e.g., Compound I) or a pharmaceutically acceptable salt, solvate, hydrate, or co-crystal thereof in an amount equivalent to about 2100 mg of the USP1 inhibitor. In some aspects, a pharmaceutical composition comprises about 2200 mg of a USP1 inhibitor (e.g., Compound I) or a pharmaceutically acceptable salt, solvate, hydrate, or co-crystal thereof in an amount equivalent to about 2200 mg of the USP1 inhibitor. In some aspects, a pharmaceutical composition comprises about 2300 mg of a USP1 inhibitor (e.g., Compound I) or a pharmaceutically acceptable salt, solvate, hydrate, or co-crystal thereof in an amount equivalent to about 2300 mg of the USP1 inhibitor. In some aspects, a pharmaceutical composition comprises about 2400 mg of a USP1 inhibitor (e.g., Compound I) or a pharmaceutically acceptable salt, solvate, hydrate, or co-crystal thereof in an amount equivalent to about 2400 mg of the USP1 inhibitor. In some aspects, a pharmaceutical composition comprises about 2500 mg of a USP1 inhibitor (e.g., Compound I) or a pharmaceutically acceptable salt, solvate, hydrate, or co-crystal thereof in an amount equivalent to about 2500 mg of the USP1 inhibitor.

In some aspects, a pharmaceutical composition comprises about 50 mg to about 2500 mg of the USP1 inhibitor or an amount of a salt, solvate, hydrate, or co-crystal thereof equivalent to about 50 mg to about 2500 mg of the USP1 inhibitor. In some aspects, a pharmaceutical composition comprises about 50 mg to about 2000 mg of the USP1 inhibitor or an amount of a salt, solvate, hydrate, or co-crystal thereof equivalent to about 50 mg to about 200 mg of the USP1 inhibitor. In some aspects, a pharmaceutical composition comprises about 50 mg to about 1500 mg of the USP1 inhibitor or an amount of a salt, solvate, hydrate, or co-crystal thereof equivalent to about 50 mg to about 1500 mg of the USP1 inhibitor. In some aspects, a pharmaceutical composition comprises about 50 mg to about 1000 mg of the USP1 inhibitor or an amount of a salt, solvate, hydrate, or co-crystal thereof equivalent to about 50 mg to about 1000 mg of the USP1 inhibitor. In some aspects, a pharmaceutical composition comprises about 100 mg to about 1000 mug of the USP1 inhibitor or an amount of a salt, solvate, hydrate, or co-crystal thereof equivalent to about 100 mg to about 1000 mg of the USP1 inhibitor. In some aspects, a pharmaceutical composition comprises about 200 mg to about 800 mg of the USP1 inhibitor or an amount of a salt, solvate, hydrate, or co-crystal thereof equivalent to about 200 mg to about 800 mg of the USP1 inhibitor. In some aspects in the paragraph above, the USP1 inhibitor is Compound I.

In some aspects, a pharmaceutical composition comprises about 75 mg to about 1000 mug of a USP1 inhibitor (e.g., Compound I or a pharmaceutically acceptable salt, solvate, hydrate, or co-crystal thereof). In some aspects, a pharmaceutical composition comprises about 75 mg of a USP1 inhibitor (e.g., Compound I or a pharmaceutically acceptable salt, solvate, hydrate, or co-crystal thereof). In some aspects, a pharmaceutical composition comprises about 100 mg of a USP1 inhibitor (e.g., Compound I or a pharmaceutically acceptable salt, solvate, hydrate, or co-crystal thereof). In some aspects, a pharmaceutical composition comprises about 150 mg of a USP1 inhibitor (e.g., Compound I or a pharmaceutically acceptable salt, solvate, hydrate, or co-crystal thereof). In some aspects, a pharmaceutical composition comprises about 200 mg of a USP1 inhibitor (e.g., Compound I or a pharmaceutically acceptable salt, solvate, hydrate, or co-crystal thereof). In some aspects, a pharmaceutical composition comprises about 250 mg of a USP1 inhibitor (e.g., Compound I or a pharmaceutically acceptable salt, solvate, hydrate, or co-crystal thereof). In some aspects, a pharmaceutical composition comprises about 300 mg of a USP1 inhibitor (e.g., Compound I or a pharmaceutically acceptable salt, solvate, hydrate, or co-crystal thereof). In some aspects, a pharmaceutical composition comprises about 350 mg of a USP1 inhibitor (e.g., Compound I or a pharmaceutically acceptable salt, solvate, hydrate, or co-crystal thereof). In some aspects, a pharmaceutical composition comprises about 400 mg of a USP1 inhibitor (e.g., Compound I or a pharmaceutically acceptable salt, solvate, hydrate, or co-crystal thereof). In some aspects, a pharmaceutical composition comprises about 450 mg of a USP1 inhibitor (e.g., Compound I or a pharmaceutically acceptable salt, solvate, hydrate, or co-crystal thereof). In some aspects, a pharmaceutical composition comprises about 500 mg of a USP1 inhibitor (e.g., Compound I or a pharmaceutically acceptable salt, solvate, hydrate, or co-crystal thereof). In some aspects, a pharmaceutical composition comprises about 550 mg of a USP1 inhibitor (e.g., Compound I or a pharmaceutically acceptable salt, solvate, hydrate, or co-crystal thereof). In some aspects, a pharmaceutical composition comprises about 600 mg of a USP1 inhibitor (e.g., Compound I or a pharmaceutically acceptable salt, solvate, hydrate, or co-crystal thereof). In some aspects, a pharmaceutical composition comprises about 650 mg of a USP1 inhibitor (e.g., Compound I or a pharmaceutically acceptable salt, solvate, hydrate, or co-crystal thereof). In some aspects, a pharmaceutical composition comprises about 700 mg of a USP1 inhibitor (e.g., Compound I or a pharmaceutically acceptable salt, solvate, hydrate, or co-crystal thereof). In some aspects, a pharmaceutical composition comprises about 750 mg of a USP1 inhibitor (e.g., Compound I or a pharmaceutically acceptable salt, solvate, hydrate, or co-crystal thereof). In some aspects, a pharmaceutical composition comprises about 800 mg of a USP1 inhibitor (e.g., Compound I or a pharmaceutically acceptable salt, solvate, hydrate, or co-crystal thereof). In some aspects, a pharmaceutical composition comprises about 850 mg of a USP1 inhibitor (e.g., Compound I or a pharmaceutically acceptable salt, solvate, hydrate, or co-crystal thereof). In some aspects, a pharmaceutical composition comprises about 900 mg of a USP1 inhibitor (e.g., Compound I or a pharmaceutically acceptable salt, solvate, hydrate, or co-crystal thereof). In some aspects, a pharmaceutical composition comprises about 950 mg of a USP1 inhibitor (e.g., Compound I or a pharmaceutically acceptable salt, solvate, hydrate, or co-crystal thereof). In some aspects, a pharmaceutical composition comprises about 1000 mg of a USP1 inhibitor (e.g., Compound I or a pharmaceutically acceptable salt, solvate, hydrate, or co-crystal thereof). In some aspects, a pharmaceutical composition comprises about 1050 mg of a USP1 inhibitor (e.g., Compound I or a pharmaceutically acceptable salt, solvate, hydrate, or co-crystal thereof). In some aspects, a pharmaceutical composition comprises about 1100 mg of a USP1 inhibitor (e.g., Compound I or a pharmaceutically acceptable salt, solvate, hydrate, or co-crystal thereof). In some aspects, a pharmaceutical composition comprises about 1150 mg of a USP1 inhibitor (e.g., Compound I or a pharmaceutically acceptable salt, solvate, hydrate, or co-crystal thereof). In some aspects, a pharmaceutical composition comprises about 1200 mg of a USP1 inhibitor (e.g., Compound I or a pharmaceutically acceptable salt, solvate, hydrate, or co-crystal thereof). In some aspects, a pharmaceutical composition comprises about 1250 mg of a USP1 inhibitor (e.g., Compound I or a pharmaceutically acceptable salt, solvate, hydrate, or co-crystal thereof). In some aspects, a pharmaceutical composition comprises about 1300 mg of a USP1 inhibitor (e.g., Compound I or a pharmaceutically acceptable salt, solvate, hydrate, or co-crystal thereof). In some aspects, a pharmaceutical composition comprises about 1350 mg of a USP1 inhibitor (e.g., Compound I or a pharmaceutically acceptable salt, solvate, hydrate, or co-crystal thereof). In some aspects, a pharmaceutical composition comprises about 1400 mg of a USP1 inhibitor (e.g., Compound I or a pharmaceutically acceptable salt, solvate, hydrate, or co-crystal thereof). In some aspects, a pharmaceutical composition comprises about 1450 mg of a USP1 inhibitor (e.g., Compound I or a pharmaceutically acceptable salt, solvate, hydrate, or co-crystal thereof). In some aspects, a pharmaceutical composition comprises about 1500 mg of a USP1 inhibitor (e.g., Compound I or a pharmaceutically acceptable salt, solvate, hydrate, or co-crystal thereof). In some aspects, a pharmaceutical composition comprises about 1550 mug of a USP1 inhibitor (e.g., Compound I or a pharmaceutically acceptable salt, solvate, hydrate, or co-crystal thereof). In some aspects, a pharmaceutical composition comprises about 1600 mg of a USP1 inhibitor (e.g., Compound I or a pharmaceutically acceptable salt, solvate, hydrate, or co-crystal thereof). In some aspects, a pharmaceutical composition comprises about 1650 mg of a USP1 inhibitor (e.g., Compound I or a pharmaceutically acceptable salt, solvate, hydrate, or co-crystal thereof). In some aspects, a pharmaceutical composition comprises about 1700 mg of a USP1 inhibitor (e.g., Compound I or a pharmaceutically acceptable salt, solvate, hydrate, or co-crystal thereof). In some aspects, a pharmaceutical composition comprises about 1750 mg of a USP1 inhibitor (e.g., Compound I or a pharmaceutically acceptable salt, solvate, hydrate, or co-crystal thereof). In some aspects, a pharmaceutical composition comprises about 1800 mg of a USP1 inhibitor (e.g., Compound I or a pharmaceutically acceptable salt, solvate, hydrate, or co-crystal thereof). In some aspects, a pharmaceutical composition comprises about 1850 mg of a USP1 inhibitor (e.g., Compound I or a pharmaceutically acceptable salt, solvate, hydrate, or co-crystal thereof). In some aspects, a pharmaceutical composition comprises about 1900 mg of a USP1 inhibitor (e.g., Compound I or a pharmaceutically acceptable salt, solvate, hydrate, or co-crystal thereof). In some aspects, a pharmaceutical composition comprises about 1950 mg of a USP1 inhibitor (e.g., Compound I or a pharmaceutically acceptable salt, solvate, hydrate, or co-crystal thereof). In some aspects, a pharmaceutical composition comprises about 2000 mg of a USP1 inhibitor (e.g., Compound I or a pharmaceutically acceptable salt, solvate, hydrate, or co-crystal thereof). In some aspects, a pharmaceutical composition comprises about 2100 mg of a USP1 inhibitor (e.g., Compound I or a pharmaceutically acceptable salt, solvate, hydrate, or co-crystal thereof). In some aspects, a pharmaceutical composition comprises about 2200 mg of a USP1 inhibitor (e.g., Compound I or a pharmaceutically acceptable salt, solvate, hydrate, or co-crystal thereof). In some aspects, a pharmaceutical composition comprises about 2300 mg of a USP1 inhibitor (e.g., Compound I or a pharmaceutically acceptable salt, solvate, hydrate, or co-crystal thereof). In some aspects, a pharmaceutical composition comprises about 2400 mg of a USP1 inhibitor (e.g., Compound I or a pharmaceutically acceptable salt, solvate, hydrate, or co-crystal thereof). In some aspects, a pharmaceutical composition comprises about 2500 mg of a USP1 inhibitor (e.g., Compound I or a pharmaceutically acceptable salt, solvate, hydrate, or co-crystal thereof).

In some aspects, such a medicament is for administration about once per day. In some aspects, such a medicament is for administration at least twice (e.g., one two consecutive days). In some aspects, such a medicament is for administration at least three times (e.g., on three consecutive days). In some aspects, such a medicament is for administration at least four times (e.g., on four consecutive days). In some aspects, such a medicament is for administration at least five times (e.g., on five consecutive days). In some aspects, such a medicament is for administration at least five times (e.g., on five consecutive days). In some aspects, such a medicament is for administration at least 10 times (e.g., on about 10 consecutive days). In some aspects, such a medicament is for administration at least 15 times (e.g., on about 15 consecutive days). In some aspects, such a medicament is for administration at least 20 times (e.g., on about 20 consecutive days). In some aspects, such a medicament is for administration at least 25 times (e.g., on about 25 consecutive days). In some aspects, such a medicament is for administration about 28 times (e.g., on about 28 consecutive days). In some aspects, such a medicament is for administration for a period of at least 3 days, or at least 4 days, or at least 5 days, or at least 10 days, or at least 15 days, or at least 20 days, or at least 25 days, or at least 1 month, or at least 2 months, or at least 3 months, or at least 4 months, or at least 5 months, or at least 6 months, or at least 7 months, or at least 8 months, or at least 9 months, or at least 10 months, or at least 11 months, or at least 1 year, or at least 18 months, or at least 2 years, or more.

In some aspects, a pharmaceutical composition is for administration about once every 21 days (about once every three weeks). In some aspects, the pharmaceutical composition is for administration at least twice (e.g., about 21 days apart). In some aspects, the pharmaceutical composition is for administration at least three times (e.g., about 21 days apart). In some aspects, the pharmaceutical composition is for administration at least four times (e.g., about 21 days apart). In some aspects, the pharmaceutical composition is for administration at least five times (e.g., about 21 days apart).

A USP1 inhibitor (e.g., Compound I or a pharmaceutically acceptable salt, solvate, hydrate, or co-crystal thereof) can be administered to a subject in the form of a raw chemical without any other components present, or such a USP1 inhibitor can also be administered as part of a pharmaceutical composition containing the inhibitor combined with a suitable pharmaceutically acceptable carrier (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Standard pharmaceutical carriers and their formulations are described in Remington's Pharmaceutical Sciences, Mack Publishing Co., Easton, PA, 19th ed. 1995.

In some aspects, the present disclosure provides a pharmaceutical composition comprising a USP1 inhibitor (e.g., Compound I or a pharmaceutically acceptable salt, solvate, hydrate, or co-crystal thereof), and a pharmaceutically acceptable carrier. In some aspects, a pharmaceutical composition comprises crystalline Form 2 of the gentisic acid co-crystal of the compound of Formula (I).

A pharmaceutical composition of the present disclosure can be orally administered in any orally acceptable dosage form including e.g., capsules, tablets, aqueous suspensions, or solutions. In some aspects, the capsule is a gelatin capsule.

For oral administration, known carriers can be included in the pharmaceutical composition. For example, microcrystalline cellulose, sodium citrate, calcium carbonate, dicalcium phosphate and glycine may be employed along with various disintegrants such as starch (preferably corn, potato or tapioca starch), methylcellulose, alginic acid and certain complex silicates, together with granulation binders such as polyvinylpyrrolidone, sucrose, gelatin and acacia, can be included in a tablet. Additionally, lubricating agents such as magnesium stearate, sodium lauryl sulfate and talc are often useful for tableting purposes. Solid compositions of a similar type may also be employed as fillers in gelatin capsules. Preferred materials in this connection include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

The pharmaceutical compositions to be used for in vivo administration can be sterile. This is readily accomplished by filtration through, e.g., sterile filtration membranes.

Pharmaceutical compositions within the scope of the present disclosure include all compositions where a USP1 inhibitor is combined with one or more pharmaceutically acceptable carriers. In some aspects, the USP1 inhibitor is present in the composition in an amount that is effective to achieve its intended therapeutic purpose.

A pharmaceutical composition of the present disclosure can be administered to any patient that may experience the beneficial effects of a Compound of the Disclosure. Foremost among such patients are mammals, e.g., humans and companion animals, although the disclosure is not intended to be so limited. In some aspects, the patient is a human. In some aspects, a pharmaceutical compositions of the present disclosure can be administered to a patient having PARP inhibitor resistant or refractory cancer. In some aspects, a pharmaceutical compositions of the present disclosure can be administered to a patient having PARP inhibitor resistant or refractory BRCA1-deficient cancer.

In some aspects, the present disclosure provides kits which comprise a USP1 inhibitor (e.g., Compound I or a pharmaceutically acceptable salt, solvate, hydrate, or co-crystal thereof) or a pharmaceutical composition comprising the same packaged in a manner that facilitates its use to practice methods of the present disclosure. In some aspects, the kit includes a USP1 inhibitor packaged in a container, such as a sealed bottle or vessel, with a label affixed to the container or included in the kit that describes use of the USP1 inhibitor or pharmaceutical composition thereof to practice the method of the disclosure. In some aspects, the USP1 inhibitor is packaged in a unit dosage form. The kit further can include a device suitable for administering the USP1 inhibitor or pharmaceutical composition thereof according to the intended route of administration. In some aspects, the present disclosure provides a kit which comprise a USP1 inhibitor or pharmaceutical composition thereof, and instructions for administering the inhibitor or composition to a patient having cancer.

In some aspects, the present disclosure provides a pharmaceutical composition comprising a USP1 inhibitor (e.g., Compound I or a pharmaceutically acceptable salt, solvate, hydrate, or co-crystal thereof) and a pharmaceutically acceptable carrier, wherein the pharmaceutical composition is for use in treating cancer (e.g., a solid tumor).

In some aspects, the present disclosure provides a pharmaceutical composition comprising a USP1 inhibitor (e.g., Compound I or a pharmaceutically acceptable salt, solvate, hydrate, or co-crystal thereof) and a pharmaceutically acceptable carrier, wherein the pharmaceutical composition is for the manufacture of a medicament for treatment of cancer (e.g., a solid tumor).

### V. Methods of Preparation of USP1 Inhibitors

USP1 inhibitors for uses and methods provided herein can be prepared in any way. Methods of synthesizing USP1 inhibitors are provided, for example, in WO2020/132269 and PCT/US2021/057072, each of which is herein incorporated by reference in its entirety.

In some aspects, a USP1 inhibitor is prepared by a method comprising a) adding a suitable amount of a compound of Formula (I) to a suitable amount of a suitable solvent system to obtain a suspension; b) stirring the suspension; and c) collecting the solid product from step b). In some aspects, a suitable pharmaceutically acceptable acid is added during step a). In some aspects, the suitable solvent system is selected from the group consisting of acetonitrile, acetone, cyclohexane, dichloromethane, dimethylacetamide, dimethyl sulfoxide, ethanol, ethyl acetate, isopropyl alcohol, isopropyl acetate, methanol, methyl ethyl ketone, 4-methyl-2-pentanone, methyl tert-butyl ether, 2-methyl tetrahydrofuran, n-heptane, n-methyl pyrrolidone, tetrahydrofuran, toluene, water, and mixtures thereof. In some aspects, the suitable solvent system is selected from the group consisting of ethyl acetate, n-heptane, and mixtures thereof.

In some aspects, a USP1 inhibitor is prepared by a method comprising a) dissolving a suitable amount of a compound of Formula (I) in a suitable amount of a suitable solvent to make a solution; b) adding a suitable amount of a suitable anti-solvent; c) adding seed crystals of a solid state form of a compound of Formula (I); d) stirring the resulting suspension; and e) collecting the solid product produced from step d). In some aspects, the method further comprises adding a suitable pharmaceutically acceptable acid during step a). In some aspects, the method further comprises adding a suitable anti-solvent after step c) and before step d). In some aspects, the suitable solvent and anti-solvent are selected from the group consisting of acetonitrile, acetone, cyclohexane, dichloromethane, dimethylacetamide, dimethyl sulfoxide, ethanol, ethyl acetate, isopropyl alcohol, isopropyl acetate, methanol, methyl ethyl ketone, 4-methyl-2-pentanone, methyl tert-butyl ether, 2-methyl tetrahydrofuran, n-heptane, n-methyl pyrrolidone, tetrahydrofuran, toluene, water, and mixtures thereof. In some aspects, the suitable solvent and anti-solvent are selected from the group consisting of ethyl acetate, n-heptane, and mixtures thereof. In some aspects, the suitable solvent is ethyl acetate. In some aspects, the suitable anti-solvent is n-heptane

In some aspects, the compound of Formula (I) is added to the suitable solvent system at a temperature of from about room temperature to about 100° C., or from about room temperature to about 75° C., or from about room temperature to about 50° C., or from about room temperature to about 40° C. In some aspects, the compound of Formula (I) is added to the suitable solvent system at about room temperature.

In some aspects, a USP1 inhibitor that is a crystalline Form 2 of a gentisic acid co-crystal of a compound of Formula (I) is prepared by a method comprising: a) adding a suitable amount of Compound I and gentisic acid to a suitable amount of a suitable solvent system at room temperature to obtain a suspension; b) stirring the suspension from step a); and c) collecting the solid product from step b). In some aspects, the suitable solvent system is selected from the group consisting of ethyl acetate, n-heptane, and mixtures thereof.

In some aspects, a USP1 inhibitor that is a crystalline Form 2 of a gentisic acid co-crystal of a compound of Formula (I) is prepared by a method comprising a) dissolving a suitable amount of Compound I and gentisic acid in a suitable amount of a suitable solvent at room temperature to make a solution; b) adding a suitable amount of a suitable anti-solvent; c) adding seed crystals of crystalline Form 2 of a gentisic acid co-crystal of Compound I of Formula (I); d) stirring the resulting suspension; and e) collecting the solid product produced from step d). In some aspects, the method further comprises adding a suitable anti-solvent after step c) and before step d). In some aspects, the suitable solvent is ethyl acetate. In some aspects, the suitable anti-solvent is n-heptane.

In some aspects, a USP1 inhibitor is a Crystalline Form 2 of a gentisic acid co-crystal of Compound I of Formula (I) prepared by any of the methods disclosed herein.

### EXAMPLES

### A. Abbreviations and Acronyms

| | |
|---|---|
| XRPD | X-ray Powder Diffraction |
| PLM | Polarized Light Microscopy |
| TGA | Thermogravimetric Analysis |
| DSC | Differential Scanning Calorimetry |
| S/AS | Solvent/Anti-solvent |
| RT | Room/ambient Temperature |
| RH | Relative Humidity |
| ACN | Acetonitrile |
| CHCl3 | Chloroform |
| DCM | Dichloromethane |
| DMAc | N,N-dimethylacetamide |
| DMSO | Dimethyl sulfoxide |
| EtOAc | Ethyl acetate |
| EtOH | Ethanol |
| H2O | Water |
| IPA | Isopropyl alcohol |
| IPAc | Isopropyl acetate |
| MeOH | Methanol |
| MEK | Methyl ethyl ketone |
| MIBK | 4-methyl-2-pentanone (methyl-iso-butyl ketone) |
| MTBE | Methyl-tert-butyl ether |
| NMP | N-methyl-2-pyrrolidone |
| THF | Tetrahydrofuran |
| 2-MeTHF | 2-Methyltetrahydrofuran |
| Cₘₐₓ | Maximum observed plasma concentration |
| Tₘₐₓ | Time to reach Cₘₐₓ |
| AUCₗₐₛₜ | Area under plasma concentration-time curve |
| | from time zero to time of last measurable |
| | concentration |
| T_{1/2} | Half-life |
| NOD/SCID | Mice homozygous for the severe combined |
| | immune deficiency |
| | spontaneous mutation |
| | Prkdc^{scid} |

### B. Experimental Methods

### Instrumental Conditions

X-ray powder diffraction (XRPD) patterns were measured on an X′Pert 3 X-ray powder diffractometer using Cu-kα radiation. Each sample was spread on the middle of a zero-background silicon holder. The tube voltage and amperage were set to 45 kV and 40 mA, respectively. A two-theta (20°) continuous scan at 46.7 seconds/step from 3° to 400

TABLE 4
XRPD Analysis Conditions.

| Parameters | Reflection Mode |
|---|---|
| Model | X′ Pert3 |
| X-Ray wavelength | Cu, kα, |
| | Kα1 (Å): 1.540598, |
| | Kα2 (Å): 1.544426 |
| | Kα2/Kα1 intensity |
| | ratio: 0.50 |
| X-Ray tube setting | 45 kV, 40 mA |
| Divergence slit | ⅛° |
| Scan mode | Continuous |
| Scan range (°2TH) | 3°-40° |
| Scan step time (s) | 46.7 |
| Step size (°2TH) | 0.0263 |
| Test time | 5 min 4 s |

Thermogravimetric analysis (TGA) data was collected using a TA Q5000 and Discovery TGA 5500 TGA from TA Instruments. TGA analysis conditions are shown in Table 5 below.

TABLE 5
TGA Analysis Conditions.

| Parameters | TGA |
|---|---|
| Method | Ramp |
| Sample pan | Aluminum, open |
| Temperature | RT-350° C. |
| Heating rate | 10° C./min |
| Purge gas | N² |

Differential scanning calorimetry (DSC) data was collected using a TA Q2000 DSC from TA Instruments. DSC analysis conditions are shown in the Table 6 below.

TABLE 6
DSC Analysis Conditions.

| Parameters | DSC |
|---|---|
| Method | Ramp |
| Sample pan | Aluminum, |
| | crimped |
| Temperature | 25° C.-300° C. |
| Heating rate | 2, 10, 20° C./min |
| Purge gas | N² |

Polarized light microscopy (PLM) images were captured with a ZEISS Scope A1 microscope.

### Example 1: Preparation and Characterization of Crystalline Form A

### A. Preparation of the Compound of Formula (I)

6-(4-cyclopropyl-6-methoxypyrimidin-5-yl)-1-(4-(4-(trifluoromethyl)-1H-imidazol-2-yl)benzyl)-1H-pyrazolo[3,4-d]pyrimidine was prepared according to the procedures disclosed in WO 2020/132269, which is herein incorporated by reference in its entirety.

To an ice cooled solution of 6-(4-cyclopropyl-6-methoxypyrimidin-5-yl)-1-(4-(4-(trifluoromethyl)-1H-imidazol-2-yl)benzyl)-1H-pyrazolo[3,4-d]pyrimidine (1 eq) in dimethylformamide (5 mL) was added sodium hydride (60% dispersion in mineral oil) (1.2 eq) portion wise, and the reaction mixture was stirred at same temperature for 10 min. To the resulting reaction mixture was added a 2-iodopropane (1.20 eq) and stirring was continued at room temperature for 16 hours. Progress of the reaction was monitored by thin layer chromatography (TLC) and liquid chromatography-mass spectrometry (LCMS). After completion, the reaction mixture was diluted with water (50 mL) and extracted with ethyl acetate (2×200 mL). The combined organic layer was dried over anhydrous sodium sulfate, filtered, and concentrated under reduced pressure. The crude compounds were purified by preparatory high performance liquid chromatography (HPLC) to afford 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I). The purified compound was then recrystallized in heptane and ethyl acetate using methods known to those skilled in the art.

### B. Characterization

6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I), obtained as discussed above, was subjected to XRPD, TGA, and DSC analysis using the conditions discussed above. The resulting XRPD pattern, DSC profile, and TGA profile are shown in FIGS. 1-2, respectively, and the XRPD peaks are shown in Table 1, above.

Based on the XRPD pattern shown in FIG. 1 and the XRPD peaks shown in Table 1 (a and b), as well as the crystal structure and DSC and TGA profiles, 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I), obtained as discussed above, was determined to be a crystalline hydrate and was named crystalline Form A.

### C. Crystal Structure Determination

About 2.9 mg of crystalline Form A was added to a 3 mL glass vial with 0.5 mL DCM/n-heptane (1:4, v/v) solvent mixture. The mixture was then shaken by an ultrasonic cleaner to accelerate dissolution. The resulting suspension was filtered and the obtained clear solution was transferred to a clean 4-mL shell vial (44.6 mm×14.65 mm). The shell vial was sealed by a PE-Plug with one pinhole. The shell vial was then placed in a fume hood at room temperature for slow evaporation. After 1 day of slow evaporation, block-like crystals were observed.

A suitable single crystal with good diffraction quality was selected from the block-like crystal samples and was wrapped with Paratone-N (an oil based cryoprotectant). The crystal was mounted on a mylar loop in a random orientation and immersed in a stream of nitrogen at 120 K. Preliminary examination and data collection were performed on a Rigaku XtaLAB Synergy R (CuKα radiation, X=1.54184 Å) diffractometer and analyzed with the CrysAlisPro (V1.171.40.19a, Rigaku, 2018) software package. Cell parameters and an orientation matrix for data collection were retrieved and refined (T-vector Dirax algorithm) by CrysAlisPro (V1.171.40.19a, Rigaku, 2018) software using the setting angles of 49260 reflections in the range 3.5630<θ<75.668°. The data were collected to a minimum diffraction angle (0) of 3.591° and a maximum diffraction angle (θ) of 76.0110 at 120 K. The final point group completeness is 100%. The mean I/σ of the data is 69.7 and the highest resolution is truncated at 0.79 Å.

Frames were integrated with CrysAlisPro (V1.171.40.19a, Rigaku, 2018). A total of 57,364 reflections were collected, of which 5292 were unique. Lorentz and polarization corrections were applied to the data. An empirical absorption correction was performed using CrysAlisPro (V1.171.40.19a, Rigaku, 2018) using spherical harmonicas implemented in SCALE3 ABSPACK. The absorption coefficient μ of this material is 0.883 mm-1 at this wavelength (=1.542 Å) and the minimum and maximum transmissions are 0.94414 and 1.0000, respectively. The agreement factor for the averaging was 5.19% based on intensity.

The structure was solved in the space group P21/c with the ShelXT structure solution program using Intrinsic Phasing and refined with ShelXL (Version 2018 3) refinement package using full-matrix least-squares on F² contained in OLEX2. All non-hydrogen atoms were refined anisotropically. The hydrogen atoms were calculated geometrically and refined using the riding model.

A calculated XRPD pattern was generated for copper (“Cu”) radiation using Mercury program and the atomic coordinates, space group, and unit cell parameters from the single crystal structure. Crystal structure representations were generated by Olex2 and Diamond. The atomic thermal displacement ellipsoids drawing was generated by ORTEP-III

A suitable single crystal was separated from the block-like crystals and selected for single-crystal X-ray diffraction data collection. The crystal system of the single crystal was monoclinic and the space group is P2₁/c. Crystallographic data and the refinement parameters are shown in Table 7.

TABLE 7
Crystallographic data of crystalline Form A

| | |
|---|---|
| Empirical formula | C₂₇H₂₅F₃N₈O•0.42(H₂O) |
| Formula weight | 541.75 |
| Temperature | 119.99(10) K |
| Wavelength | CuKα (λ = 1.54184 Å) |
| Crystal system, space group | Monoclinic, P2₁/c |
| Unit cell dimensions | a = 12.05400(10) Å |
| | b = 8.77450(10) Å |
| | c = 24.83660(10) Å |
| | α = 90° |
| | β = 97.6260(10)° |
| | γ = 90° |
| Volume | 2603.68(4) Å³ |
| Z, CalMolated density | 4, 1.382 g/cm³ |
| Absorption coefficient | 0.883 mm⁻¹ |
| F(000) | 1128.0 |
| Crystal size | 0.12 × 0.11 × 0.1 mm³ |
| 2 Theta range for data collection | 7.182 to 152.022 |
| Limiting indices | −13 ≤ h ≤ 15 |
| | −10 ≤ k ≤ 11 |
| | −31 ≤ l ≤ 31 |
| Reflections | 57364/5292 [Rᵢₙₜ = 0.0519, |
| collected/Independent reflections | R_{sigma} = 0.0143] |
| Refinement method | Full-matrix least-squares on F² |
| Completeness | 100% |
| Data / restraints / parameters | 5292/16/408 |
| Goodness-of-fit on F² | 1.084 |
| Final R indices [I ≥ 2sigma(I)] | R₁ = 0.0463, wR₂ = 0.1142 |
| Final R indices [all data] | R₁ = 0.0467, wR₂ = 0.1144 |
| Largest diff. peak and hole | 0.50/−0.41 e · Å⁻³ |

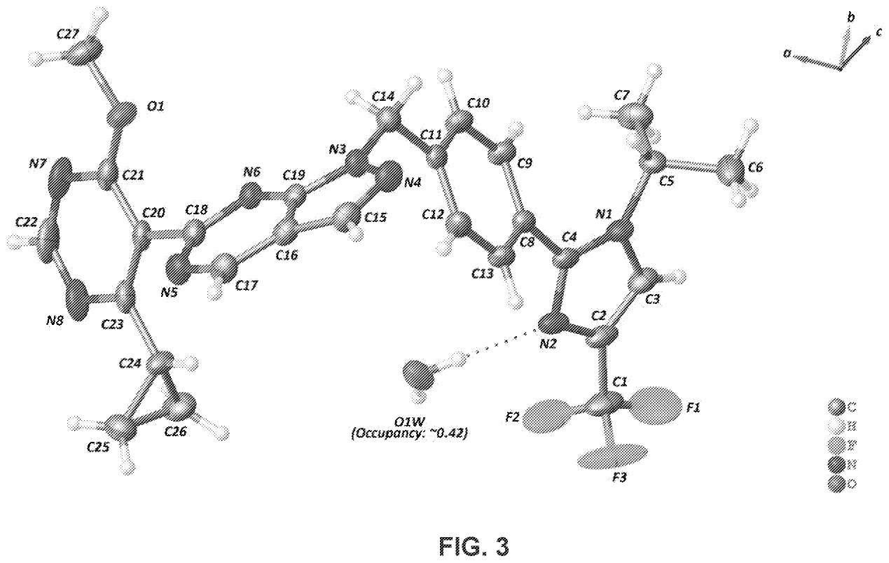

As shown in FIG. 3, the asymmetric unit of the single crystal structure is comprised of one freebase compound of Formula (I) molecule and a non-integer number of water molecules, which suggested that crystalline Form A is hydrate. The number of water molecule in the asymmetric unit was freely refined to be 0.42, according to the thermal parameters.

### Example 2: Preparation and Characterization of Crystalline Form 2

### A. Preparation

Method A: Approximately 40 mg of crystalline Form A and 11.6 mg of gentisic acid were combined with 0.5 mL EtOAc/n-Heptane to obtain a suspension. The suspension was stirred at room temperature for 2 days, and the solid material was isolated by vacuum filtration and dried under vacuum at room temperature. The resulting solid material can be used as “seeds” to prepare crystalline Form 1 at larger scales.

Method B: Approximately 500 mg of crystalline Form A and 11.6 mg of gentisic acid were combined with 7 mL EtOAc to obtain a solution. 5.0 mL n-heptane was then added dropwise, along with 10.2 mg of gentisic acid co-crystal “seeds” prepared according to method A, and finally 8.0 mL n-heptane to obtain a suspension. The suspension was stirred at room temperature for two days. The resulting solid material was isolated by vacuum filtration and dried under vacuum at room temperature.

### B. Characterization

The resulting solid material was subjected to XRPD, TGA, and DSC analysis using the conditions discussed above. The resulting XRPD pattern, DSC profile, and TGA profile are shown in FIGS. 2 and 3, respectively, and the XRPD peaks are shown in Table 1, above.

Based on the XRPD pattern shown in FIG. 4 and the XRPD peaks shown in Table 2, as well as the crystal structure and DSC and TGA profiles, the obtained solid material was determined to be a crystalline anhydrate of a gentisic acid co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula (I) and was named crystalline Form 2.

### C. Crystal Structure Determination

About 3.0 mg of crystalline Form 2 was added to a 3 mL glass vial with 0.5 mL THF/n-heptane (2:5, v/v) solvent mixture. The mixture was then shaken by an ultrasonic cleaner to accelerate dissolution. The resulting suspension was filtered and the obtained clear solution was transferred to a clean 4-mL shell vial (44.6 mm×14.65 mm). The shell vial was sealed by a PE-Plug with one pinhole. The shell vial was then placed in a fume hood at room temperature for slow evaporation. After 6 days of slow evaporation, block-like crystals were observed.

A suitable single crystal with good diffraction quality was selected out from the block-like crystal sample and was wrapped with Paratone-N (an oil based cryoprotectant). The crystal was mounted on a mylar loop in a random orientation and immersed in a stream of nitrogen at 120 K. Preliminary examination and data collection were performed on a Rigaku XtaLAB Synergy R (CuKα radiation, λ=1.54184 Å) diffractometer and analyzed with the CrysAlisPro (V1.171.40.19a, Rigaku, 2018) software package. Cell parameters and an orientation matrix for data collection were retrieved and refined (T-vector Dirax algorithm) by CrysAlisPro (V1.171.40.19a, Rigaku, 2018) software using the setting angles of 50053 reflections in the range 3.5390<θ<75.936°. The data were collected to a minimum diffraction angle (θ) of 3.7650 and a maximum diffraction angle (θ) of 76.018° at 120 K. The final point group completeness is 100%. The mean I/a of the data is 75.8 and the highest resolution is truncated at 0.79 Å.

Frames were integrated with CrysAlisPro (V1.171.40.19a, Rigaku, 2018). A total of 58850 reflections were collected, of which 6566 were unique. Lorentz and polarization corrections were applied to the data. An empirical absorption correction was performed using CrysAlisPro (V1.171.40.19a, Rigaku, 2018) using spherical harmonicas implemented in SCALE3 ABSPACK. The absorption coefficient μ of this material is 0.927 mm¹ at this wavelength (λ=1.542 Å) and the minimum and maximum transmissions are 0.75213 and 1.0000, respectively. The agreement factor for the averaging was 3.94% based on intensity.

The structure was solved in the space group P2₁/c with the ShelXT structure solution program using Intrinsic Phasing and refined with ShelXL (Version 2018 3) refinement package using full-matrix least-squares on F² contained in OLEX2. All non-hydrogen atoms were refined anisotropically. The hydrogen atoms (H2) connected with the oxygen atoms (O2) was determined and refine freely based on the Fourier Map. Other hydrogen atoms were calculated geometrically and refined using the riding model.

A calculated XRPD pattern was generated for copper (“Cu”) radiation using Mercury program and the atomic coordinates, space group, and unit cell parameters from the single crystal structure. Crystal structure representations were generated by Olex2 and Diamond. The thermal ellipsoids drawing was generated by ORTEP-III

A suitable single crystal was separated and selected out from the block-like crystals and selected for single-crystal x-ray diffraction data collection. The crystal system of the single crystal was determined to be monoclinic and the space group was determined to be P21/c. Crystallographic data and the refinement parameters are listed in Table 8.

TABLE 8
Crystallographic data of crystalline Form 2

| | |
|---|---|
| Empirical formula | C₃₄H₃₁F₃N₈O₅ |
| Formula weight | 688.67 |
| Temperature | 120.00(10) K |
| Wavelength | CuKα (λ = 1.54184 Å) |
| Crystal system, space group | Monoclinic, P2₁/c |
| Unit cell dimensions | a = 11.11290(10) Å |
| | b = 12.35560(10) Å |
| | c = 24.0484(2) Å |
| | α = 90° |
| | β = 102.4840(10)° |
| | γ = 90° |
| Volume | 3223.93(5) Å³ |
| Z, CalMolated density | 4, 1.419 g/cm³ |
| Absorption coefficient | 0.927 mm⁻¹ |
| F(000) | 1432.0 |
| Crystal size | 0.31 × 0.15 × 0.11 mm³ |
| 2 Theta range for data | 7.53 to 152.036 |
| collection | |
| Limiting indices | −13 ≤ h ≤ 13 |
| | −15 ≤ k ≤ 14 |
| | −30 ≤ l ≤ 29 |
| Reflections | 58850/6566 [Rᵢₙₜ = 0.0394, |
| collected/Independent | R_{sigma} = 0.0132] |
| reflections | |
| Refinement method | Full-matrix least-squares on |
| | F² |
| Completeness | 100% |
| Data / restraints / | 6566/20/500 |
| parameters | |
| Goodness-of-fit on F² | 1.160 |
| Final R indices [I ≥ | R₁ = 0.0426, wR₂ = 0.0973 |
| 2sigma(I)] | |
| Final R indices [all data] | R₁ = 0.0427, wR₂ = 0.0976 |
| Largest diff. peak and hole | 0.27/−0.23 e · Å⁻³ |

As shown in FIG. 6, the asymmetric unit of the single crystal structure is comprised of one neutral compound of Formula (I) molecule and one gentisic acid neutral molecule, which indicated that crystalline Form 2 was actually a co-crystal of the starting compound with gentisic acid.

### Example 3: Mouse Pharmacokinetic Studies

Mouse pharmacokinetic (PK) studies were conducted using Crystalline Form A of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, as disclosed herein and Crystalline Form 2 of a gentisic acid co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, as disclosed herein.

The PK studies consisted of single dose oral exposure studies, which were conducted in female NOD/SCID mice (approximately 6 to 8 weeks, and 20-30 g at the time of study) using a compound dose of 300 mg/kg and a dosing volume of 10 mL/kg. The mice were fasted overnight prior to dosing. Animals had free access to food and water post-dosing.

Blood was sampled serially from the dorsal metatarsal vein at pre-dose, 0.25, 0.5, 1, 2, 4, 8, 24, 48 (crystalline Form 2 only), and 72 hours (crystalline Form 2 only) post PO dosing. Approximately 0.03 mL blood was collected at each time point and centrifuged at 4000 G for 5 minutes at 4° C. to provide plasma. The plasma samples were then stored in a freezer at −75±15° C. prior to LC-MS/MS analysis.

Concentrations of each compound in the plasma samples were then analyzed using an LC-MS/MS method. WinNonlin (Phoenix™, version 6.1) or other similar software was used for PK calculations. The following PK parameters were calculated, whenever possible from the plasma concentration versus time data: Cₘₐₓ, Tₘₐₓ, T_{1/2}, AUC_{inf}, and AUCₗₐₛₜ. The PK data were described using descriptive statistics such as mean with standard deviation.

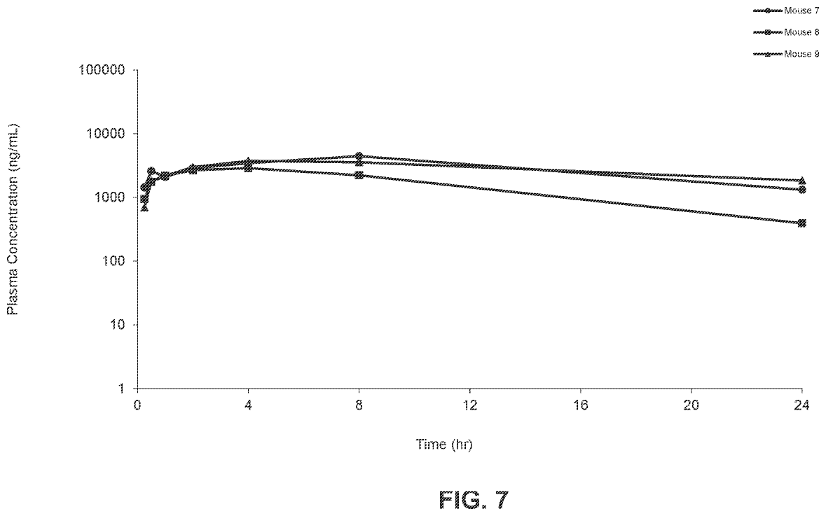

The results for crystalline form A are shown in Table 9 below and in FIG. 7.

TABLE 9
Summary of Crystalline Form A PK Parameters

| PK Parameters | Unit | Mouse 7 | Mouse 8 | Mouse 9 | Mean | SD | CV(%) |
|---|---|---|---|---|---|---|---|
| T_{1/2} | h | NA | 6.79 | 18.6 | 12.7 | NA | NA |
| Tₘₐₓ | h | 8.00 | 4.00 | 4.00 | 5.33 | 2.31 | 43.3 |
| Cₘₐₓ | ng/mL | 4450 | 2890 | 3750 | 3697 | 781 | 21.1 |
| AUCₗₐₛₜ | h*ng/mL | 72653 | 40678 | 68658 | 60663 | 17422 | 28.7 |

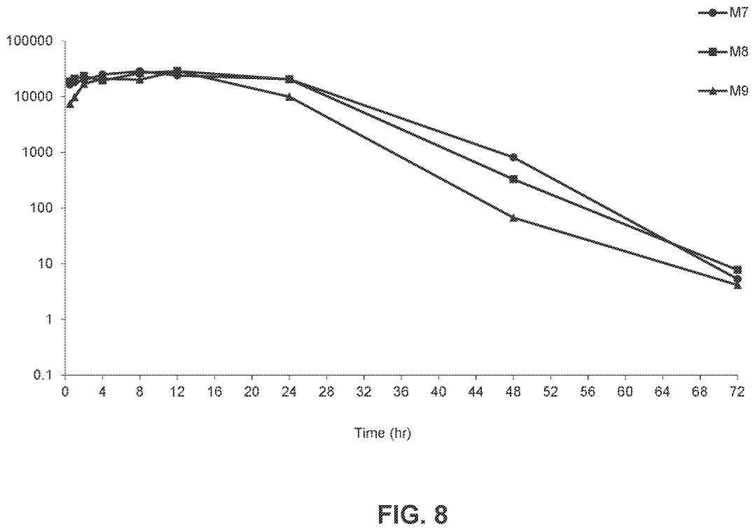

The results for crystalline Form 2 are shown in Table 10 below and in FIG. 8.

TABLE 10
Summary of Crystalline Form 2 PK Parameters

| PK Parameters | Unit | Mouse 7 | Mouse 8 | Mouse 9 | Mean | SD | CV(%) |
|---|---|---|---|---|---|---|---|
| T_{1/2} | h | 4.03 | 4.22 | 4.27 | 4.17 | 0.13 | 3.12 |
| Tₘₐₓ | h | 8.00 | 12.0 | 12.0 | 10.7 | 2.3 | 21.7 |
| Cₘₐₓ | ng/mL | 28800 | 28900 | 29000 | 28900 | 100 | 0.346 |
| AUCₗₐₛₜ | h*ng/mL | 827348 | 835713 | 594288 | 752450 | 137036 | 18.2 |

### Example 4: Distribution of Crystalline Form 2 in Mouse Brain and Plasma

Female NOD SCID mice of an age between 6-8 weeks and a body weight range of 18-22 g were purchased from Beijing Anikeeper Biotech Co, Ltd. Animals were habituated to the environment for at least 7 days prior to study initiation. Mice were dosed with Crystalline Form 2 at either 100 mg/kg or 300 mg/kg dose levels via oral gavage for 28 days. After 24 hours following 27 doses of compound, three mice were euthanized via CO₂. After four hours following 28 doses of compound, three mice were euthanized via CO₂. At each time point the whole brain was collected. In addition, approximately 0.03 mL of blood was collected at each time point and centrifuged at 4000G for 5 minutes at 4° C. to provide plasma. Samples were then stored in a freezer at −75±15° C. prior to LC-MS/MS analysis. Concentrations of compound in brain homogenates (ng/g) and plasma (ng/ml) were then analyzed using an LC-MS/MS method. The PK data were described using descriptive statistics such as mean with standard deviation.

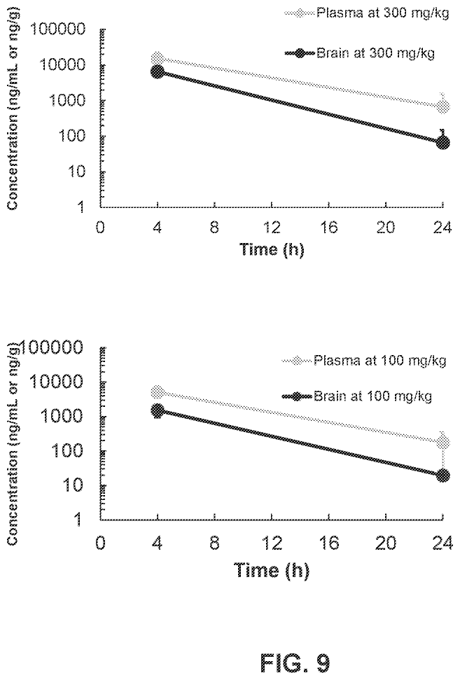

As shown in Table 11 and FIG. 9, measurable drug level was observed in the brain tissue. There was no apparent accumulation of the drug in the brain after repeated dosing.

TABLE 11
Summary of brain penetration of crystalline
Form 2 in NOD SCID mice

Crystalline Form 2

| Parameters | 4 h (Day 28) | 24 h (Day 28) |
|---|---|---|
| 100 mg/kg | | |
| Brain Conc. (ng/g) | 1525 ± 559 | 19.5 |
| Plasma Conc. (ng/mL) | 5040 ± 694 | 178 ± 165 |
| B/P Ratio | 0.306 ± 0.125 | 0.091 ± 0.014 |
| 300 mg/kg | | |
| Brain Conc. (ng/g) | 6500 ± 1050 | 66.1 ± 81.7 |
| Plasma Conc. (ng/mL) | 15333 ± 2318 | 667 ± 852 |
| B/P Ratio | 0.424 ± 0.018 | 0.102 ± 0.007 |

### Example 5. Distribution of Crystalline Form 2 in Rat Brain and Plasma

Male and female Sprague Dawley rats were purchased from Beijing Anikeeper Biotech Co, Ltd. Animals were habituated to the environment prior to study initiation. Rats were dosed with single dose of 100 mg/kg crystalline Form 2 via oral gavage. The rats were fasted overnight prior to dosing. Animals had access to food from 2 hours post dose and free access to water throughout the study. Approximately 0.2 mL of blood was sampled via jugular vein at each time point and centrifuged at 4000G for 5 minutes at 4° C. to provide plasma. Samples were then stored in a freezer at −75±15° C. prior to LC-MS/MS analysis. Whole brain was collected after hemoperfusion and snap frozen on dry ice prior to storage at −75±15° C. Prior to LC-MS/MS analysis whole brains were weighed and homogenized with water by tissue weight (g) to water volume (ml) ratio 1:3 before analysis. Actual concentration is the measured value multiplied by the dilution factor. The PK data were described using descriptive statistics such as mean with standard deviation.

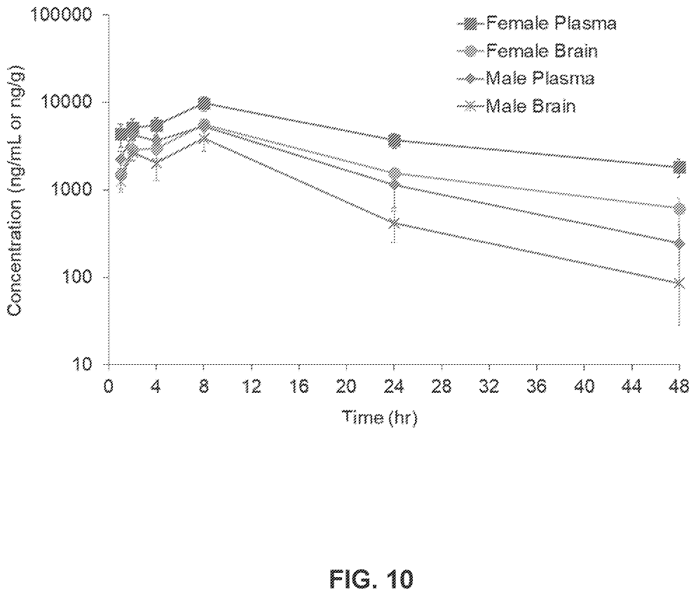

As shown in Table 12 and FIG. 10, measurable drug level was observed in the brain tissue. A similar elimination profile was also observed between plasma and brain tissue, suggesting a rapid equilibrium between systemic circulation and brain tissue.

TABLE 12
Summary of brain penetration of crystalline
Form 2 in SD male and female rats

Crystalline Form 2

| Parameters | Plasma | Brain |
|---|---|---|
| Male | | |
| Cₘₐₓ (ng/mL or ng/g) | 5297 | 3903 |
| tₘₐₓ (h) | 8.00 | 8.00 |
| t_{1/2} (h) | 9.13 | 7.43 |
| AUC_{0-∞} (ng · h/mL or ng · h/g) | 101618 | 60596 |

| | |
|---|---|
| B/P Conc. Ratio at tₘₐₓ | 0.731 |

| | | |
|---|---|---|
| Female | | |
| Cₘₐₓ (ng/mL or ng/g) | 9760 | 5613 |
| tₘₐₓ (h) | 8.00 | 8.00 |
| t_{1/2} (h) | 16.9 | 12.8 |
| AUC_{0-∞} (ng · h/mL or ng · h/g) | 266414 | 120933 |

| | |
|---|---|
| B/P Conc. Ratio at tₘₐₓ | 0.580 |

### Example 6: Phase 1 Administration of USP1 Inhibitors for Treatment of Solid Tumors

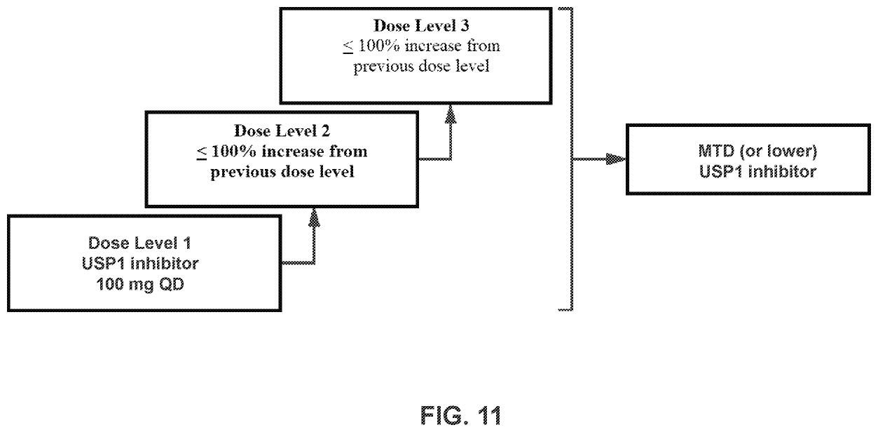

A Phase 1 dose escalating study of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (provided in the form of a gentisate co-crystal) is performed in patients with advanced solid tumors. The dose escalation schema is shown in FIG. 11. The starting dose is 100 mg once a day (QD) on a 28-day cycle. The dose is administered orally. A first group of patients (e.g., 3 patients) is enrolled at this starting dose, and patients are enrolled in the next higher dose level if none of the first group of patients experiences a dose limiting toxicity (DLT; DLTs are provided in Table 1) in the first cycle (28 days). If one DLT is observed among the previous group of patients, more patients (e.g., 3 more patients) will be treated at the same dose level. If no DLTs are seen among these patients, patients will then be enrolled in the next highest dose level. If 2 or more DLTs are found among 6 patients, then the maximum tolerated dose (MTD) is defined as the previous dose level tested. If 2 or 3 out of 3 patients experience DLTs at any dose level, the previous level is the MTD.

TABLE 1
Dose-Limiting Toxicities (DLTs)

| Toxicity | Any of the following: |
|---|---|
| Hematology | Grade 4 neutropenia (absolute neutrophil count [ANC] <500/μL) lasting |
| | more than 7 days |
| | Grade 4 febrile neutropenia of any duration (single oral |
| | temperature >38.3° C. or ≥38° C. for more than 1 hour) |
| | Grade 4 thrombocytopenia (platelets <25,000/μL) of any duration |
| | ≥Grade 3 thrombocytopenia (platelets <50,000/μL) with clinically |
| | significant bleeding or any requirement for platelet transfusion |
| Hepatic | ≥Grade 3 total bilirubin (except Gilbert's disease) |
| | ≥Grade 3 alanine aminotransferase/aspartate aminotransferase (ALT/AST) |
| | lasting more than 7 days in patients with ≤Grade 1 ALT/AST at study |
| | entry |
| | Tripling of ALT/AST lasting more than 7 days in patients with Grade 2 |
| | ALT/AST at study entry. |
| Gastrointestinal | ≥Grade 3 vomiting or Grade 3 nausea of more than 72 hours duration |
| | despite optimal anti-emetic supportive care |
| | ≥Grade 3 diarrhea of more than 72 hours duration despite optimal anti- |
| | diarrheal supportive care |
| | ≥Grade 3 gastric ulcer, necrosis or perforation |
| Other | ≥Grade 3 adrenal insufficiency |
| | Any dose delay of the USP1 inhibitor more than 3 weeks due to drug- |
| | related toxicity |
| | Other ≥Grade 3 non-hematological toxicities may be considered a DLT as |
| | determined by the Investigator in conjunction with the safety review |
| | committee (SRC) |
| Exceptions to DLT | ≥Grade 3 anorexia |
| criteria | ≥Grade 3 fatigue lasting more than 7 days |
| | ≥Grade 3 nausea and/or vomiting, or diarrhea (unless lasting more than 72 |
| | hours despite optimal medical management) |
| | ≥Grade 3 constipation lasting more than 5 days |
| | ≥Grade 3 metabolic abnormalities (hypokalemia, hypomagnesemia, |
| | hypocalcemia, hypophosphatemia) that recovers to Grade 2 or less within |
| | 48 hours with or without medical management |

Patients that generally meet the following “initial criteria” are eligible for inclusion in the Phase 1 study:
Age 18 years or older;
Life expectancy of ≥12 weeks;
Recovered to ≤Grade 1 or baseline toxicity (except alopecia) from prior therapy (per National Cancer Institute (NCI)— Common Terminology Criteria for Adverse Events (CTCAE) v5.0);
Eastern Cooperative Oncology Group performance status 0 or 1;
Adequate bone marrow function at baseline;
Adequate organ function at baseline;
Does not require ongoing treatment with strong or moderate CYP3A4 inhibitors or inducers.

Patients generally also meet the following “additional criteria” to participate:
Measurable disease or non-measurable per RECIST v1.1;
Histologically or cytologically confirmed locally advanced (unresectable) or metastatic solid tumors who meet one of the following criteria: (a) Relapsed or progressed through standard therapy; (b) have a disease for which no standard effective therapy exists; or (c) not a candidate for standard effective therapy.

Patients that meet any of the following “exclusion criteria” generally do not participate in the Phase 1 study:
Prior anti-cancer treatment including
   Chemotherapy or small molecule-targeted therapy <2 weeks prior to first dose of study treatment;
   Any antibody therapy <5 half-lives from first dose of study treatment (or 4 weeks since last therapy, whichever is the shortest);
   PD-1 or PD-L1 therapy <4 weeks from first dose of study treatment;
   Invasive surgery requiring general anesthesia <30 days from first dose of study treatment;
   Chemotherapy with nitrosoureas or mitomycin C, <45 days from first dose of study treatment; and
   Radiation therapy (including radiofrequency ablation)<4 weeks prior to initiation of study treatment (Note: Prior stereotactic body radiation therapy or local palliative radiation is allowed <2 weeks prior to first dose of study treatment);
Grade 2 or greater toxicity, except alopecia related to any prior treatment (i.e., chemotherapy, targeted therapy, radiation, or surgery);
Prolongation of QT/QTc interval (QTc interval >480 msec) using the Frederica method of QTc analysis;
Seropositive for human immunodeficiency virus (HIV) 1 or 2 or acquired immunodeficiency syndrome (AIDS) or active infection with hepatitis B virus or hepatitis C virus (HCV) (Note: Patients with positive HCV antibody may be eligible if HCV ribonucleic acid [RNA] is undetectable on a quantitative HCV RNA assay, following discussion with the Medical Monitor);
Women who are pregnant or nursing;
Other severe, acute, or chronic medical condition or laboratory abnormality that may increase the risk associated with study participation or study drug administration, or that may interfere with the interpretation of the study results, and would the patient inappropriate for the study;

The objective response rate (ORR per Response Evaluation Criteria in Solid Tumors version 1.1 (RECIST v1.1) Investigator assessment), progression-free survival (PFS), duration of response (DOR per RECIST v1.1 Investigator assessment), and time to response (TTR per RECIST v1.1 Investigator assessment) are assessed to demonstrate that 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine is effective in treating solid tumors.

### Example 7: Phase 2 Administration of USP1 Inhibitors for Treatment of Solid Tumors

A Phase 2 dose expansion study of the USP1 inhibitor 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (provided in the form of a gentisate co-crystal) is performed in patients with advanced solid tumors. In this study, the USP1 inhibitor is administered until disease progression, investigator decision, withdrawal of consent, or other protocol-specified reason for discontinuation of study treatment. No intra-patient dose escalation occurs in this portion of the study. The USP1 inhibitor is tested at the MTD (or lower dose).

Patients that generally meet the “initial criteria” as discussed in Example 1 and have measurable disease per RECIST v1.1 are eligible for inclusion in the Phase 2 study.

The ORR per RECIST v.1.1 by independent central radiologic review (ICRR) is assessed to demonstrate that 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine is effective in treating solid tumors. In addition, the PFS, DOR, clinical benefit rate, and TTR (all per RECIST v.1.1 per ICRR) are assessed to demonstrate that 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine is effective in treating solid tumors. Furthermore, overall survival (OS) is assessed to demonstrate that 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine is effective in treating solid tumors.

### Exemplary Instances of the Invention

Exemplary instances of the invention are provided below.

In one instance (I1), a method of treating a solid tumor in a human patient comprises administering to the patient about 1000 mg to about 2250 mg of the ubiquitin-specific-processing protease 1 (USP1) inhibitor 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound I), or a salt, solvate, hydrate, or co-crystal thereof in an amount equivalent to about 1000 mg to about 2250 mg of Compound I. In one instance (I2) of I1, about 1000 mg of the USP1 inhibitor or an amount of the salt, solvate, hydrate, or co-crystal thereof equivalent to about 1000 mg of the USP1 inhibitor is administered. In one instance (I3) of I1, about 1050 mg of the USP1 inhibitor or an amount of the salt, solvate, hydrate, or co-crystal thereof equivalent to about 1050 mg of the USP1 inhibitor is administered. In one instance (I4) of I1, about 1100 mg of the USP1 inhibitor or an amount of the salt, solvate, hydrate, or co-crystal thereof equivalent to about 1100 mg of the USP1 inhibitor is administered. In one instance (I5) of I1, about 1150 mg of the USP1 inhibitor or an amount of the salt, solvate, hydrate, or co-crystal thereof equivalent to about 1150 mg of the USP1 inhibitor is administered. In one instance (I6) of I1, about 1200 mg of the USP1 inhibitor or an amount of the salt, solvate, hydrate, or co-crystal thereof equivalent to about 1200 mg of the USP1 inhibitor is administered. In one instance (I7) of I1, about 1250 mg of the USP1 inhibitor or an amount of the salt, solvate, hydrate, or co-crystal thereof equivalent to about 1250 mg of the USP1 inhibitor is administered. In one instance (I8) of I1, about 1300 mg of the USP1 inhibitor or an amount of the salt, solvate, hydrate, or co-crystal thereof equivalent to about 1300 mg of the USP1 inhibitor is administered. In one instance (I9) of I1, about 1350 mg of the USP1 inhibitor or an amount of the salt, solvate, hydrate, or co-crystal thereof equivalent to about 1350 mg of the USP1 inhibitor is administered. In one instance (I10) of I1, about 1400 mg of the USP1 inhibitor or an amount of the salt, solvate, hydrate, or co-crystal thereof equivalent to about 1400 mg of the USP1 inhibitor is administered. In one instance (I11) of I1, about 1450 mg of the USP1 inhibitor or an amount of the salt, solvate, hydrate, or co-crystal thereof equivalent to about 1450 mg of the USP1 inhibitor is administered. In one instance (I12) of I1, about 1500 mg of the USP1 inhibitor or an amount of the salt, solvate, hydrate, or co-crystal thereof equivalent to about 1500 mg of the USP1 inhibitor is administered. In one instance (I13) of I1, about 1550 mg of the USP1 inhibitor or an amount of the salt, solvate, hydrate, or co-crystal thereof equivalent to about 1550 mg of the USP1 inhibitor is administered. In one instance (I14) of I1, about 1600 mg of the USP1 inhibitor or an amount of the salt, solvate, hydrate, or co-crystal thereof equivalent to about 1600 mg of the USP1 inhibitor is administered. In one instance (I15) of I1, about 1650 mg of the USP1 inhibitor or an amount of the salt, solvate, hydrate, or co-crystal thereof equivalent to about 1650 mg of the USP1 inhibitor is administered. In one instance (I16) of I1, about 1700 mg of the USP1 inhibitor or an amount of the salt, solvate, hydrate, or co-crystal thereof equivalent to about 1700 mg of the USP1 inhibitor is administered. In one instance (I17) of I1, about 1750 mg of the USP1 inhibitor or an amount of the salt, solvate, hydrate, or co-crystal thereof equivalent to about 1750 mg of the USP1 inhibitor is administered. In one instance (I18) of I1, about 1800 mg of the USP1 inhibitor or an amount of the salt, solvate, hydrate, or co-crystal thereof equivalent to about 1800 mg of the USP1 inhibitor is administered. In one instance (I19) of I1, about 1850 mg of the USP1 inhibitor or an amount of the salt, solvate, hydrate, or co-crystal thereof equivalent to about 1850 mg of the USP1 inhibitor is administered. In one instance (I20) of I1, about 1900 mg of the USP1 inhibitor or an amount of the salt, solvate, hydrate, or co-crystal thereof equivalent to about 1900 mg of the USP1 inhibitor is administered. In one instance (I21) of I1, about 1950 mg of the USP1 inhibitor or an amount of the salt, solvate, hydrate, or co-crystal thereof equivalent to about 1950 mg of the USP1 inhibitor is administered. In one instance (I22) of I1, about 2000 mg of the USP1 inhibitor or an amount of the salt, solvate, hydrate, or co-crystal thereof equivalent to about 2000 mg of the USP1 inhibitor is administered. In one instance (I23) of I1, about 2050 mg of the USP1 inhibitor or an amount of the salt, solvate, hydrate, or co-crystal thereof equivalent to about 2050 mg of the USP1 inhibitor is administered. In one instance (I24) of I1, about 2100 mg of the USP1 inhibitor or an amount of the salt, solvate, hydrate, or co-crystal thereof equivalent to about 2100 mg of the USP1 inhibitor is administered. In one instance (I25) of I1, about 2150 mg of the USP1 inhibitor or an amount of the salt, solvate, hydrate, or co-crystal thereof equivalent to about 2150 mg of the USP1 inhibitor is administered. In one instance (I26) of I1, about 2200 mg of the USP1 inhibitor or an amount of the salt, solvate, hydrate, or co-crystal thereof equivalent to about 2200 mg of the USP1 inhibitor is administered. In one instance (I27) of I1, about 2250 mg of the USP1 inhibitor or an amount of the salt, solvate, hydrate, or co-crystal thereof equivalent to about 2250 mg of the USP1 inhibitor is administered.

In one instance (I28), a method of treating a solid tumor in a human patient comprises administering to the patient about 75 mg to about 1000 mg of the ubiquitin-specific-processing protease 1 (USP1) inhibitor 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound I), or a salt, solvate, hydrate, or co-crystal thereof in an amount equivalent to about 75 mg to about 1000 mg of Compound I. In one instance (I29) of 128, about 75 mg of the USP1 inhibitor or an amount of the salt, solvate, hydrate, or co-crystal thereof equivalent to about 75 mg of the USP1 inhibitor is administered. In one instance (I30) of 128, about 100 mg of the USP1 inhibitor or an amount of the salt, solvate, hydrate, or co-crystal thereof equivalent to about 100 mg of the USP1 inhibitor is administered. In one instance (I31) of 128, about 150 mg of the USP1 inhibitor or an amount of the salt, solvate, hydrate, or co-crystal thereof equivalent to about 150 mg of the USP1 inhibitor is administered. In one instance (I32) of 128, about 200 mg of the USP1 inhibitor or an amount of the salt, solvate, hydrate, or co-crystal thereof equivalent to about 200 mg of the USP1 inhibitor is administered. In one instance (I33) of 128, about 250 mg of the USP1 inhibitor or an amount of the salt, solvate, hydrate, or co-crystal thereof equivalent to about 250 mg of the USP1 inhibitor is administered. In one instance (I34) of 128, about 300 mg of the USP1 inhibitor or an amount of the salt, solvate, hydrate, or co-crystal thereof equivalent to about 300 mg of the USP1 inhibitor is administered. In one instance (I35) of 128, about 350 mg of the USP1 inhibitor or an amount of the salt, solvate, hydrate, or co-crystal thereof equivalent to about 350 mg of the USP1 inhibitor is administered. In one instance (I36) of 128, about 400 mg of the USP1 inhibitor or an amount of the salt, solvate, hydrate, or co-crystal thereof equivalent to about 400 mg of the USP1 inhibitor is administered. In one instance (I37) of 128, about 450 mg of the USP1 inhibitor or an amount of the salt, solvate, hydrate, or co-crystal thereof equivalent to about 450 mg of the USP1 inhibitor is administered. In one instance (I38) of 128, about 500 mg of the USP1 inhibitor or an amount of the salt, solvate, hydrate, or co-crystal thereof equivalent to about 500 mg of the USP1 inhibitor is administered. In one instance (I39) of 128, about 550 mg of the USP1 inhibitor or an amount of the salt, solvate, hydrate, or co-crystal thereof equivalent to about 550 mg of the USP1 inhibitor is administered. In one instance (I40) of 128, about 600 mg of the USP1 inhibitor or an amount of the salt, solvate, hydrate, or co-crystal thereof equivalent to about 600 mg of the USP1 inhibitor is administered. In one instance (I41) of 128, about 650 mg of the USP1 inhibitor or an amount of the salt, solvate, hydrate, or co-crystal thereof equivalent to about 650 mg of the USP1 inhibitor is administered. In one instance (I42) of 128, about 700 mg of the USP1 inhibitor or an amount of the salt, solvate, hydrate, or co-crystal thereof equivalent to about 700 mg of the USP1 inhibitor is administered. In one instance (143) of 128, about 750 mg of the USP1 inhibitor or an amount of the salt, solvate, hydrate, or co-crystal thereof equivalent to about 750 mg of the USP1 inhibitor is administered. In one instance (I44) of 128, about 800 mg of the USP1 inhibitor or an amount of the salt, solvate, hydrate, or co-crystal thereof equivalent to about 800 mg of the USP1 inhibitor is administered. In one instance (I45) of 128, about 850 mg of the USP1 inhibitor or an amount of the salt, solvate, hydrate, or co-crystal thereof equivalent to about 850 mg of the USP1 inhibitor is administered. In one instance (I46) of 128, about 900 mg of the USP1 inhibitor or an amount of the salt, solvate, hydrate, or co-crystal thereof equivalent to about 900 mg of the USP1 inhibitor is administered. In one instance (I47) of 128, about 950 mg of the USP1 inhibitor or an amount of the salt, solvate, hydrate, or co-crystal thereof equivalent to about 950 mg of the USP1 inhibitor is administered. In one instance (I48) of 128, about 1000 mg of the USP1 inhibitor or an amount of the salt, solvate, hydrate, or co-crystal thereof equivalent to about 1000 mg of the USP1 inhibitor is administered.

In one instance (I49) of any one of I1 to 148, the USP1 inhibitor or the salt, solvate, hydrate, or co-crystal thereof is administered about once daily.

In one instance (I50) of any one of I1 to 148, the USP1 inhibitor or the salt, solvate, hydrate, or co-crystal thereof is administered as a divided dose over the course of a day, optionally wherein the administration is divided into two doses, wherein the two doses are the same or wherein the two doses are different.

In one instance (I51) of any one of I1 to I50, the USP1 inhibitor or the salt, solvate, hydrate, or co-crystal thereof is administered orally.

In one instance (I52) of any one of I1 to I51, the cancer is ovarian cancer. In one instance (I53) of any one of I1 to 152, the ovarian cancer is serous ovarian cancer. In one instance (I54) of any one of I1 to I51, the cancer is breast cancer. In one instance (I55) of any one of 152 to 154, the patient has previously received platinum-based chemotherapy. In one instance (I56) of any one of 152 to 154, the cancer is platinum-sensitive. In one instance (I57) of any one of 152 to 154, the cancer is platinum-resistant. In one instance (I58) of any one of 154 to 157, the breast cancer is human epidermal growth factor receptor 2 (HER2) negative. In one instance (I59) of any one of 154 to 158, the breast cancer is triple negative breast cancer.

In one instance (I60) of any one of I1 to 159, the cancer has a mutation in BRCA1. In one instance (161) of any one of I1 to 160, the cancer has a mutation in BRCA2.

In one instance (I62) of any one of 154 to 161, the breast cancer is germline breast cancer susceptibility gene mutation (gBRCAm) breast cancer. In one instance (I63) of any one of 154 to 162, the breast cancer is metastatic.

In one instance (I64) of any one of I1 to 163, the patient has previously received treatment with at least one systemic chemotherapy for metastatic disease.

In one instance (I65) of any one of I1 to I51, the cancer is selected from the group consisting of a DNA damage repair pathway deficient cancer, a homologous-recombination deficient cancer, a cancer comprising cancer cells with a mutation in a gene encoding p53, and a cancer comprising cancer cells with a loss of function mutation in a gene encoding p53.

In one instance (I66) of any one of I1 to I51, the cancer is selected from the group consisting of lung cancer, non-small cell lung cancer (NSCLC), colon cancer, bladder cancer, osteosarcoma, ovarian cancer, skin cancer, and breast cancer.

In one instance (I67) of any one of I1 to 166, the cancer comprises cancer cells with elevated levels of RAD18 protein and/or RAD18 mRNA. In one instance (I68) of any one of I1 to 167, the cancer comprises cancer cells with elevated levels of RAD51 protein and/or RAD51 mRNA. In one instance (I69) of 168, the elevated levels of RAD51 are elevated RAD51 protein foci levels. In one instance (I70) of 168, at least 10% of cells that are in the S/G2 phase of the cell cycle in a sample obtained from the cancer are RAD51-positive.

In one instance (I71) of any one of I1 to 170, the cancer is recurrent.

In one instance (I72) of any one of I1 to 171, the patient has previously received treatment with a PARP inhibitor (PARPi). In one instance (I73) of any one of I1 to 172, the cancer is a PARP inhibitor resistant or refractory cancer.

In one instance (I74) of any one of I1 to 173, the Compound I is administered as a co-crystal with a pharmaceutically acceptable acid. In one instance (I75) of 174, the pharmaceutically acceptable acid is gentisic acid.

In one instance (I76) of any one of I1 to 175, the USP1 inhibitor or the salt, solvate, hydrate, or co-crystal thereof is administered at least three times, at least 4 times, or at least 5 times.

In one instance (I77) of any one of I1 to 175, the USP1 inhibitor or the salt, solvate, hydrate, or co-crystal thereof is administered at least 10 days, at least 15 days, at least 20 days, at least 25 days, at least 1 month, at least 2 months, at least 3 months, at least 4 months, at least 5 months, at least 6 months, at least 7 months, at least 8 months, at least 9 months, at least 10 months, at least 11 months, at least 1 year, at least 18 months, or at least 2 years.

In one instance (I78) a USP1 inhibitor 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound I) or the salt, solvate, hydrate, or co-crystal thereof is for use in the method of any one of 11 to 177.

1. A method of treating a solid tumor in a human patient, comprising administering to the patient about 75 mg to about 2250 mg of an ubiquitin-specific-processing protease 1 (USP1) inhibitor 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound I), or a salt, solvate, hydrate, or co-crystal thereof in an amount equivalent to about 75 mg to about 2250 mg of the Compound I.

2. The method of claim 1, wherein about 1000 mg to about 2250 mg of the Compound I, or an amount of the salt, solvate, hydrate, or co-crystal thereof equivalent to about 1000 mg to about 2250 mg of the Compound I is administered.

3. The method of claim 1, wherein about 1000 mg of the Compound I or an amount of the salt, solvate, hydrate, or co-crystal thereof equivalent to about 1000 mg of the Compound I is administered

4. The method of claim 1, wherein about 75 mg to about 1000 mg of the Compound I or an amount of the salt, solvate, hydrate, or co-crystal thereof equivalent to about 75 mg to about 1000 mg of the Compound I is administered.

5. The method of claim 1 wherein about 100 mg of the Compound I or an amount of the salt, solvate, hydrate, or co-crystal thereof equivalent to about 100 mg of the Compound I is administered.

6. The method of claim 1, wherein the Compound I or the salt, solvate, hydrate, or co-crystal thereof is administered about once daily.

7. The method of claim 1, wherein the Compound I or the salt, solvate, hydrate, or co-crystal thereof is administered as a divided dose over the course of a day.

8. The method of claim 1, wherein the Compound I or the salt, solvate, hydrate, or co-crystal thereof is administered orally.

9-16. (canceled)

17. The method of claim 1, wherein the cancer has a mutation in BRCA1.

18. The method of claim 1, wherein the cancer has a mutation in BRCA2.

19-21. (canceled)

22. The method of claim 1, wherein the cancer is selected from the group consisting of a DNA damage repair pathway deficient cancer, a homologous-recombination deficient cancer, a cancer comprising cancer cells with a mutation in a gene encoding p53, and a cancer comprising cancer cells with a loss of function mutation in a gene encoding p53.

23. The method of claim 1, wherein the cancer is selected from the group consisting of lung cancer, non-small cell lung cancer (NSCLC), colon cancer, bladder cancer, osteosarcoma, ovarian cancer, skin cancer, and breast cancer.

24. The method of claim 1, wherein the cancer comprises cancer cells with elevated levels of RAD18 protein and/or RAD18 mRNA.

25. The method of claim 1, wherein the cancer comprises cancer cells with elevated levels of RAD51 protein and/or RAD51 mRNA.

26. The method of claim 25, wherein the elevated levels of RAD51 are elevated RAD51 protein foci levels.

27. The method of claim 25, wherein at least 10% of cells that are in the S/G2 phase of the cell cycle in a sample obtained from the cancer are RAD51-positive.

28. (canceled)

29. The method of claim 1, wherein the patient has previously received treatment with a PARP inhibitor (PARPi).

30. The method of claim 1, wherein the cancer is a PARP inhibitor resistant or refractory cancer.

31. The method of claim 1, wherein the Compound I is administered as a cocrystal with a pharmaceutically acceptable acid.

32. The method of claim 31, wherein the pharmaceutically acceptable acid is gentisic acid.

33-35. (canceled)
